SEQUENCE LISTING (234 sequences)

SEQ ID NO: 1 - protein, 4 aa, synthetic construct
  REGION 1..4: note = Description of sequence: recognition sequence
GNRS

SEQ ID NO: 2 - protein, 4 aa, synthetic construct
  REGION 1..4: note = Description of sequence: recognition sequence
GFKG

SEQ ID NO: 3 - protein, 4 aa, synthetic construct
  REGION 1..4: note = Description of sequence: recognition sequence
AAGQ

SEQ ID NO: 4 - protein, 4 aa, synthetic construct
  REGION 1..4: note = Description of sequence: recognition sequence
ALKH

SEQ ID NO: 5 - protein, 4 aa, synthetic construct
  REGION 1..4: note = Description of sequence: recognition sequence
EFAR

SEQ ID NO: 6 - protein, 4 aa, synthetic construct
  REGION 1..4: note = Description of sequence: recognition sequence
GHKF

SEQ ID NO: 7 - protein, 4 aa, synthetic construct
  REGION 1..4: note = Description of sequence: recognition sequence
GGKU

SEQ ID NO: 8 - protein, 4 aa, synthetic construct
  REGION 1..4: note = Description of sequence: recognition sequence
AAAR

SEQ ID NO: 9 - protein, 4 aa, synthetic construct
  REGION 1..4: note = Description of sequence: recognition sequence
ALKN

SEQ ID NO: 10 - protein, 4 aa, synthetic construct
  REGION 1..4: note = Description of sequence: recognition sequence
FGSV

SEQ ID NO: 11 - protein, 4 aa, synthetic construct
  REGION 1..4: note = Description of sequence: recognition sequence
GHYA

SEQ ID NO: 12 - protein, 4 aa, synthetic construct
  REGION 1..4: note = Description of sequence: recognition sequence
GFSF

SEQ ID NO: 13 - protein, 4 aa, synthetic construct
  REGION 1..4: note = Description of sequence: recognition sequence
AURU

SEQ ID NO: 14 - protein, 4 aa, synthetic construct
  REGION 1..4: note = Description of sequence: recognition sequence
AAKF

SEQ ID NO: 15 - protein, 4 aa, synthetic construct
  REGION 1..4: note = Description of sequence: recognition sequence
RNYN

SEQ ID NO: 16 - protein, 4 aa, synthetic construct
  REGION 1..4: note = Description of sequence: recognition sequence
GGKA

SEQ ID NO: 17 - protein, 4 aa, synthetic construct
  REGION 1..4: note = Description of sequence: recognition sequence
AALK

SEQ ID NO: 18 - protein, 4 aa, synthetic construct
  REGION 1..4: note = Description of sequence: recognition sequence
ASKA

SEQ ID NO: 19 - protein, 4 aa, synthetic construct
  REGION 1..4: note = Description of sequence: recognition sequence
SFKG

SEQ ID NO: 20 - protein, 4 aa, synthetic construct
  REGION 1..4: note = Description of sequence: recognition sequence
MIHE

SEQ ID NO: 21 - protein, 4 aa, synthetic construct
  REGION 1..4: note = Description of sequence: recognition sequence
GGRA

SEQ ID NO: 22 - protein, 4 aa, synthetic construct
  REGION 1..4: note = Description of sequence: recognition sequence
AHKA

SEQ ID NO: 23 - protein, 4 aa, synthetic construct
  REGION 1..4: note = Description of sequence: recognition sequence
AFKN

SEQ ID NO: 24 - protein, 4 aa, synthetic construct
  REGION 1..4: note = Description of sequence: recognition sequence
SHKG

SEQ ID NO: 25 - protein, 4 aa, synthetic construct
  REGION 1..4: note = Description of sequence: recognition sequence
GNFK

SEQ ID NO: 26 - protein, 4 aa, synthetic construct
  REGION 1..4: note = Description of sequence: recognition sequence
ALAR

SEQ ID NO: 27 - protein, 4 aa, synthetic construct
  REGION 1..4: note = Description of sequence: recognition sequence
AGVR

SEQ ID NO: 28 - protein, 4 aa, synthetic construct
  REGION 1..4: note = Description of sequence: recognition sequence
EFTR

SEQ ID NO: 29 - protein, 4 aa, synthetic construct
  REGION 1..4: note = Description of sequence: recognition sequence
GAGK

SEQ ID NO: 30 - protein, 4 aa, synthetic construct
  REGION 1..4: note = Description of sequence: recognition sequence
GRAU

SEQ ID NO: 31 - protein, 4 aa, synthetic construct
  REGION 1..4: note = Description of sequence: recognition sequence
GFHG

SEQ ID NO: 32 - protein, 4 aa, synthetic construct
  REGION 1..4: note = Description of sequence: recognition sequence
AAGK

SEQ ID NO: 33 - protein, 4 aa, synthetic construct
  REGION 1..4: note = Description of sequence: recognition sequence
PARV

SEQ ID NO: 34 - protein, 4 aa, synthetic construct
  REGION 1..4: note = Description of sequence: recognition sequence
GYLS

SEQ ID NO: 35 - protein, 4 aa, synthetic construct
  REGION 1..4: note = Description of sequence: recognition sequence
GHNK

SEQ ID NO: 36 - protein, 4 aa, synthetic construct
  REGION 1..4: note = Description of sequence: recognition sequence
GQUY

SEQ ID NO: 37 - protein, 4 aa, synthetic construct
  REGION 1..4: note = Description of sequence: recognition sequence
UAKS

SEQ ID NO: 38 - protein, 4 aa, synthetic construct
  REGION 1..4: note = Description of sequence: recognition sequence
ERKG

SEQ ID NO: 39 - protein, 4 aa, synthetic construct
  REGION 1..4: note = Description of sequence: recognition sequence
GAUR

SEQ ID NO: 40 - protein, 4 aa, synthetic construct
  REGION 1..4: note = Description of sequence: recognition sequence
GYGI

SEQ ID NO: 41 - protein, 4 aa, synthetic construct
  REGION 1..4: note = Description of sequence: recognition sequence
GAFI

SEQ ID NO: 42 - protein, 4 aa, synthetic construct
  REGION 1..4: note = Description of sequence: recognition sequence
UUKN

SEQ ID NO: 43 - protein, 4 aa, synthetic construct
  REGION 1..4: note = Description of sequence: recognition sequence
EIGR

SEQ ID NO: 44 - protein, 4 aa, synthetic construct
  REGION 1..4: note = Description of sequence: recognition sequence
GYAF

SEQ ID NO: 45 - protein, 4 aa, synthetic construct
  REGION 1..4: note = Description of sequence: recognition sequence
GNKY

SEQ ID NO: 46 - protein, 4 aa, synthetic construct
  REGION 1..4: note = Description of sequence: recognition sequence
GNKG

SEQ ID NO: 47 - protein, 4 aa, synthetic construct
  REGION 1..4: note = Description of sequence: recognition sequence
UASR

SEQ ID NO: 48 - protein, 4 aa, synthetic construct
  REGION 1..4: note = Description of sequence: recognition sequence
RHTR

SEQ ID NO: 49 - protein, 4 aa, synthetic construct
  REGION 1..4: note = Description of sequence: recognition sequence
GHFG

SEQ ID NO: 50 - protein, 4 aa, synthetic construct
  REGION 1..4: note = Description of sequence: recognition sequence
GLKI

SEQ ID NO: 51 - protein, 4 aa, synthetic construct
  REGION 1..4: note = Description of sequence: recognition sequence
GHAG

SEQ ID NO: 52 - protein, 4 aa, synthetic construct
  REGION 1..4: note = Description of sequence: recognition sequence
UMKG

SEQ ID NO: 53 - protein, 4 aa, synthetic construct
  REGION 1..4: note = Description of sequence: recognition sequence
RHFI

SEQ ID NO: 54 - protein, 4 aa, synthetic construct
  REGION 1..4: note = Description of sequence: recognition sequence
GFGY

SEQ ID NO: 55 - protein, 4 aa, synthetic construct
  REGION 1..4: note = Description of sequence: recognition sequence
GGRV

SEQ ID NO: 56 - protein, 4 aa, synthetic construct
  REGION 1..4: note = Description of sequence: recognition sequence
GVYG

SEQ ID NO: 57 - protein, 4 aa, synthetic construct
  REGION 1..4: note = Description of sequence: recognition sequence
SLKM

SEQ ID NO: 58 - protein, 4 aa, synthetic construct
  REGION 1..4: note = Description of sequence: recognition sequence
RTNL

SEQ ID NO: 59 - protein, 4 aa, synthetic construct
  REGION 1..4: note = Description of sequence: recognition sequence
GMLY

SEQ ID NO: 60 - protein, 4 aa, synthetic construct
  REGION 1..4: note = Description of sequence: recognition sequence
GYUN

SEQ ID NO: 61 - protein, 4 aa, synthetic construct
  REGION 1..4: note = Description of sequence: recognition sequence
GALK

SEQ ID NO: 62 - protein, 4 aa, synthetic construct
  REGION 1..4: note = Description of sequence: recognition sequence
SLYP

SEQ ID NO: 63 - protein, 4 aa, synthetic construct
  REGION 1..4: note = Description of sequence: recognition sequence
KPAK

SEQ ID NO: 64 - protein, 4 aa, synthetic construct
  REGION 1..4: note = Description of sequence: recognition sequence
GSSK

SEQ ID NO: 65 - protein, 4 aa, synthetic construct
  REGION 1..4: note = Description of sequence: recognition sequence
GFIG

SEQ ID NO: 66 - protein, 4 aa, synthetic construct
  REGION 1..4: note = Description of sequence: recognition sequence
AHKK

SEQ ID NO: 67 - protein, 4 aa, synthetic construct
  REGION 1..4: note = Description of sequence: recognition sequence
SUKG

SEQ ID NO: 68 - protein, 4 aa, synthetic construct
  REGION 1..4: note = Description of sequence: recognition sequence
YEML

SEQ ID NO: 69 - protein, 4 aa, synthetic construct
  REGION 1..4: note = Description of sequence: recognition sequence
GAIK

SEQ ID NO: 70 - protein, 4 aa, synthetic construct
  REGION 1..4: note = Description of sequence: recognition sequence
GGKF

SEQ ID NO: 71 - protein, 4 aa, synthetic construct
  REGION 1..4: note = Description of sequence: recognition sequence
AHNK

SEQ ID NO: 72 - protein, 4 aa, synthetic construct
  REGION 1..4: note = Description of sequence: recognition sequence
SFNK

SEQ ID NO: 73 - protein, 4 aa, synthetic construct
  REGION 1..4: note = Description of sequence: recognition sequence
YRDU

SEQ ID NO: 74 - protein, 4 aa, synthetic construct
  REGION 1..4: note = Description of sequence: recognition sequence
GUGR

SEQ ID NO: 75 - protein, 4 aa, synthetic construct
  REGION 1..4: note = Description of sequence: recognition sequence
GRAL

SEQ ID NO: 76 - protein, 4 aa, synthetic construct
  REGION 1..4: note = Description of sequence: recognition sequence
AUKL

SEQ ID NO: 77 - protein, 4 aa, synthetic construct
  REGION 1..4: note = Description of sequence: recognition sequence
SAKK

SEQ ID NO: 78 - protein, 4 aa, synthetic construct
  REGION 1..4: note = Description of sequence: recognition sequence
FRGK

SEQ ID NO: 79 - protein, 4 aa, synthetic construct
  REGION 1..4: note = Description of sequence: recognition sequence
GYSK

SEQ ID NO: 80 - protein, 4 aa, synthetic construct
  REGION 1..4: note = Description of sequence: recognition sequence
GRGA

SEQ ID NO: 81 - protein, 4 aa, synthetic construct
  REGION 1..4: note = Description of sequence: recognition sequence
AMIK

SEQ ID NO: 82 - protein, 4 aa, synthetic construct
  REGION 1..4: note = Description of sequence: recognition sequence
SNKG

SEQ ID NO: 83 - protein, 4 aa, synthetic construct
  REGION 1..4: note = Description of sequence: recognition sequence
LUFN

SEQ ID NO: 84 - protein, 4 aa, synthetic construct
  REGION 1..4: note = Description of sequence: recognition sequence
GMGK

SEQ ID NO: 85 - protein, 5 aa, synthetic construct
  REGION 1..5: note = Description of sequence: Linker sequence
  MOD_RES 1: note = bAla
  MOD_RES 2: note = bAla
AARRM

SEQ ID NO: 86 - protein, 6 aa, synthetic construct
  REGION 1..6: note = Description of sequence: cloning construct
  MOD_RES <1: note = Histidine bound to expression vector
  MOD_RES >1: note = Histidine bound to expression vector
HHHHHH

SEQ ID NO: 87 - protein, 110 aa, synthetic construct
  REGION 1..110: note = Description of sequence: coding sequence product
KTSNTIRVLLPNQEWTVVKVRNGMSLHDSLMKALKRHGLQPESSAVFRLLHEHKGKKARLDWNTDAASLIGEELQVDFLDHVPLTTHNFARKTFLKLGIHRDGSSGSSLE

SEQ ID NO: 88 - protein, 116 aa, synthetic construct
  REGION 1..116: note = Description of sequence: coding sequence product
KTSNTIRVLLPNQEWTVVKVRNGMSLHDSLMKALKRHGLQPESSAVFRLLHEHKGKKARLDWNTDAASLIGEELQVDFLDHVPLTTHNFARKTFLKLGIHRDGSSGSSLEHHHHHH

SEQ ID NO: 89 - protein, 7 aa, synthetic construct
  REGION 1..7: note = Description of sequence: cloning construct component
MPCGHKP

SEQ ID NO: 90 - protein, 123 aa, synthetic construct
  REGION 1..123: note = Description of sequence: cloning construct
  MOD_RES <1: note = Methionine bound to expression vector
  MOD_RES >1: note = Histidine bound to expression vector
MPCGHKPKTSNTIRVLLPNQEWTVVKVRNGMSLHDSLMKALKRHGLQPESSAVFRLLHEHKGKKARLDWNTDAASLIGEELQVDFLDHVPLTTHNFARKTFLKLGIHRDGSSGSSLEHHHHHH

SEQ ID NO: 91 - DNA, 32 bp, synthetic construct
  misc_feature 1..32: note = Description of sequence: Primer
  source 1..32: mol_type = other DNA
ggtcacaaaccgaagaccagcaataccatccg

SEQ ID NO: 92 - DNA, 34 bp, synthetic construct
  misc_feature 1..34: note = Description of sequence: Primer
  source 1..34: mol_type = other DNA
cggtttgtgaccgcacggcatatgtatatctcct

SEQ ID NO: 93 - DNA, 40 bp, synthetic construct
  misc_feature 1..40: note = Description of sequence: Primer
  source 1..40: mol_type = other DNA
ggttcttctggttcttctaagaccagcaataccatccgtg

SEQ ID NO: 94 - DNA, 35 bp, synthetic construct
  misc_feature 1..35: note = Description of sequence: Primer
  source 1..35: mol_type = other DNA
agaagaaccagaagaacccggtttgtgaccgcacg

SEQ ID NO: 95 - protein, 122 aa, synthetic construct
  REGION 1..122: note = Description of sequence: Protein substrate
PCGHKPKTSNTIRVLLPNQEWTVVKVRNGMSLHDSLMKALKRHGLQPESSAVFRLLHEHKGKKARLDWNTDAASLIGEELQVDFLDHVPLTTHNFARKTFLKLGIHRDGSSGSSLEHHHHHH

SEQ ID NO: 96 - protein, 128 aa, synthetic construct
  REGION 1..128: note = Description of sequence: Protein substrate
PCGHKPGSSGSSKTSNTIRVLLPNQEWTVVKVRNGMSLHDSLMKALKRHGLQPESSAVFRLLHEHKGKKARLDWNTDAASLIGEELQVDFLDHVPLTTHNFARKTFLKLGIHRDGSSGSSLEHHHHHH

SEQ ID NO: 97 - protein, 11 aa, synthetic construct
  REGION 1..11: note = Description of sequence: Peptide library archetype
  VARIANT 2..5: note = alanine, arginine, asparagine, aspartic acid, glutamine, glutamic acid, glycine, histidine, isoleucine, leucine, lysine, phenylalanine, proline, serine, threonine, tryptophan, tyrosine, valine, Nle, bAla and Abu
PCXXXXBBRRM

SEQ ID NO: 98 - protein, 4 aa, synthetic construct
  REGION 1..4: note = Description of sequence: Peptide from library screening
GNRS

SEQ ID NO: 99 - protein, 4 aa, synthetic construct
  REGION 1..4: note = Description of sequence: Peptide from library screening
GHKF

SEQ ID NO: 100 - protein, 4 aa, synthetic construct
  REGION 1..4: note = Description of sequence: Peptide from library screening
GHYA

SEQ ID NO: 101 - protein, 4 aa, synthetic construct
  REGION 1..4: note = Description of sequence: Peptide from library screening
GGKA

SEQ ID NO: 102 - protein, 4 aa, synthetic construct
  REGION 1..4: note = Description of sequence: Peptide from library screening
GGRA

SEQ ID NO: 103 - protein, 4 aa, synthetic construct
  REGION 1..4: note = Description of sequence: Peptide from library screening
GNFK

SEQ ID NO: 104 - protein, 4 aa, synthetic construct
  REGION 1..4: note = Description of sequence: Peptide from library screening
GFKG

SEQ ID NO: 105 - skipped

SEQ ID NO: 106 - protein, 4 aa, synthetic construct
  REGION 1..4: note = Description of sequence: Peptide from library screening
GFSF

SEQ ID NO: 107 - protein, 4 aa, synthetic construct
  REGION 1..4: note = Description of sequence: Peptide from library screening
AALK

SEQ ID NO: 108 - protein, 4 aa, synthetic construct
  REGION 1..4: note = Description of sequence: Peptide from library screening
AHKA

SEQ ID NO: 109 - protein, 4 aa, synthetic construct
  REGION 1..4: note = Description of sequence: Peptide from library screening
ALAR

SEQ ID NO: 110 - protein, 4 aa, synthetic construct
  REGION 1..4: note = Description of sequence: Peptide from library screening
AAGQ

SEQ ID NO: 111 - protein, 4 aa, synthetic construct
  REGION 1..4: note = Description of sequence: Peptide from library screening
AAAR

SEQ ID NO: 112 - skipped

SEQ ID NO: 113 - protein, 4 aa, synthetic construct
  REGION 1..4: note = Description of sequence: Peptide from library screening
ASKA

SEQ ID NO: 114 - protein, 4 aa, synthetic construct
  REGION 1..4: note = Description of sequence: Peptide from library screening
AFKN

SEQ ID NO: 115 - protein, 4 aa, synthetic construct
  REGION 1..4: note = Description of sequence: Peptide from library screening
AGVR

SEQ ID NO: 116 - protein, 4 aa, synthetic construct
  REGION 1..4: note = Description of sequence: Peptide from library screening
ALKH

SEQ ID NO: 117 - protein, 4 aa, synthetic construct
  REGION 1..4: note = Description of sequence: Peptide from library screening
ALKN

SEQ ID NO: 118 - protein, 4 aa, synthetic construct
  REGION 1..4: note = Description of sequence: Peptide from library screening
AAKF

SEQ ID NO: 119 - protein, 4 aa, synthetic construct
  REGION 1..4: note = Description of sequence: Peptide from library screening
SFKG

SEQ ID NO: 120 - protein, 4 aa, synthetic construct
  REGION 1..4: note = Description of sequence: Peptide from library screening
SHKG

SEQ ID NO: 121 - protein, 4 aa, synthetic construct
  REGION 1..4: note = Description of sequence: Peptide from library screening
EFTR

SEQ ID NO: 122 - protein, 4 aa, synthetic construct
  REGION 1..4: note = Description of sequence: Peptide from library screening
EFAR

SEQ ID NO: 123 - protein, 4 aa, synthetic construct
  REGION 1..4: note = Description of sequence: Peptide from library screening
FGSV

SEQ ID NO: 124 - protein, 4 aa, synthetic construct
  REGION 1..4: note = Description of sequence: Peptide from library screening
RNYN

SEQ ID NO: 125 - skipped

SEQ ID NO: 126 - protein, 4 aa, synthetic construct
  REGION 1..4: note = Description of sequence: Peptide from library screening
GAGK

SEQ ID NO: 127 - protein, 4 aa, synthetic construct
  REGION 1..4: note = Description of sequence: Peptide from library screening
GYLS

SEQ ID NO: 128 - skipped

SEQ ID NO: 129 - protein, 4 aa, synthetic construct
  REGION 1..4: note = Description of sequence: Peptide from library screening
GYAF

SEQ ID NO: 130 - protein, 4 aa, synthetic construct
  REGION 1..4: note = Description of sequence: Peptide from library screening
GHFG

SEQ ID NO: 131 - protein, 4 aa, synthetic construct
  REGION 1..4: note = Description of sequence: Peptide from library screening
GFGY

SEQ ID NO: 132 - skipped

SEQ ID NO: 133 - protein, 4 aa, synthetic construct
  REGION 1..4: note = Description of sequence: Peptide from library screening
GSSK

SEQ ID NO: 134 - protein, 4 aa, synthetic construct
  REGION 1..4: note = Description of sequence: Peptide from library screening
GAIK

SEQ ID NO: 135 - skipped

SEQ ID NO: 136 - protein, 4 aa, synthetic construct
  REGION 1..4: note = Description of sequence: Peptide from library screening
GYSK

SEQ ID NO: 137-138 - skipped (2 entries)

SEQ ID NO: 139 - protein, 4 aa, synthetic construct
  REGION 1..4: note = Description of sequence: Peptide from library screening
GHNK

SEQ ID NO: 140 - protein, 4 aa, synthetic construct
  REGION 1..4: note = Description of sequence: Peptide from library screening
GYGI

SEQ ID NO: 141 - protein, 4 aa, synthetic construct
  REGION 1..4: note = Description of sequence: Peptide from library screening
GNKY

SEQ ID NO: 142 - protein, 4 aa, synthetic construct
  REGION 1..4: note = Description of sequence: Peptide from library screening
GLKI

SEQ ID NO: 143 - protein, 4 aa, synthetic construct
  REGION 1..4: note = Description of sequence: Peptide from library screening
GGRV

SEQ ID NO: 144 - skipped

SEQ ID NO: 145 - protein, 4 aa, synthetic construct
  REGION 1..4: note = Description of sequence: Peptide from library screening
GFIG

SEQ ID NO: 146 - protein, 4 aa, synthetic construct
  REGION 1..4: note = Description of sequence: Peptide from library screening
GGKF

SEQ ID NO: 147 - protein, 4 aa, synthetic construct
  REGION 1..4: note = Description of sequence: Peptide from library screening
GRAL

SEQ ID NO: 148 - protein, 4 aa, synthetic construct
  REGION 1..4: note = Description of sequence: Peptide from library screening
GRGA

SEQ ID NO: 149 - protein, 4 aa, synthetic construct
  REGION 1..4: note = Description of sequence: Peptide from library screening
GFHG

SEQ ID NO: 150 - skipped

SEQ ID NO: 151 - protein, 4 aa, synthetic construct
  REGION 1..4: note = Description of sequence: Peptide from library screening
GAFI

SEQ ID NO: 152 - protein, 4 aa, synthetic construct
  REGION 1..4: note = Description of sequence: Peptide from library screening
GNKG

SEQ ID NO: 153 - protein, 4 aa, synthetic construct
  REGION 1..4: note = Description of sequence: Peptide from library screening
GHAG

SEQ ID NO: 154 - protein, 4 aa, synthetic construct
  REGION 1..4: note = Description of sequence: Peptide from library screening
GVYG

SEQ ID NO: 155 - protein, 4 aa, synthetic construct
  REGION 1..4: note = Description of sequence: Peptide from library screening
GALK

SEQ ID NO: 156 - protein, 4 aa, synthetic construct
  REGION 1..4: note = Description of sequence: Peptide from library screening
AHKK

SEQ ID NO: 157 - protein, 4 aa, synthetic construct
  REGION 1..4: note = Description of sequence: Peptide from library screening
AHNK

SEQ ID NO: 158-159 - skipped (2 entries)

SEQ ID NO: 160 - protein, 4 aa, synthetic construct
  REGION 1..4: note = Description of sequence: Peptide from library screening
AAGK

SEQ ID NO: 161-165 - skipped (5 entries)

SEQ ID NO: 166 - protein, 4 aa, synthetic construct
  REGION 1..4: note = Description of sequence: Peptide from library screening
SLYP

SEQ ID NO: 167 - skipped

SEQ ID NO: 168 - protein, 4 aa, synthetic construct
  REGION 1..4: note = Description of sequence: Peptide from library screening
SFNK

SEQ ID NO: 169 - protein, 4 aa, synthetic construct
  REGION 1..4: note = Description of sequence: Peptide from library screening
SAKK

SEQ ID NO: 170 - protein, 4 aa, synthetic construct
  REGION 1..4: note = Description of sequence: Peptide from library screening
SNKG

SEQ ID NO: 171 - protein, 4 aa, synthetic construct
  REGION 1..4: note = Description of sequence: Peptide from library screening
PARV

SEQ ID NO: 172 - protein, 4 aa, synthetic construct
  REGION 1..4: note = Description of sequence: Peptide from library screening
ERKG

SEQ ID NO: 173 - protein, 4 aa, synthetic construct
  REGION 1..4: note = Description of sequence: Peptide from library screening
EIGR

SEQ ID NO: 174 - protein, 4 aa, synthetic construct
  REGION 1..4: note = Description of sequence: Peptide from library screening
RHTR

SEQ ID NO: 175 - protein, 4 aa, synthetic construct
  REGION 1..4: note = Description of sequence: Peptide from library screening
RHFI

SEQ ID NO: 176 - protein, 4 aa, synthetic construct
  REGION 1..4: note = Description of sequence: Peptide from library screening
RTNL

SEQ ID NO: 177 - protein, 4 aa, synthetic construct
  REGION 1..4: note = Description of sequence: Peptide from library screening
KPAK

SEQ ID NO: 178-179 - skipped (2 entries)

SEQ ID NO: 180 - protein, 4 aa, synthetic construct
  REGION 1..4: note = Description of sequence: Peptide from library screening
FRGK

SEQ ID NO: 181 - skipped

SEQ ID NO: 182 - protein, 4 aa, synthetic construct
ADDR

SEQ ID NO: 183 - protein, 4 aa, synthetic construct
APPE

SEQ ID NO: 184-185 - skipped (2 entries)

SEQ ID NO: 186 - protein, 4 aa, synthetic construct
DKPK

SEQ ID NO: 187 - protein, 4 aa, synthetic construct
DFVD

SEQ ID NO: 188-189 - skipped (2 entries)

SEQ ID NO: 190 - protein, 4 aa, synthetic construct
HDET

SEQ ID NO: 191 - protein, 4 aa, synthetic construct
HIDS

SEQ ID NO: 192 - protein, 4 aa, synthetic construct
HAYV

SEQ ID NO: 193 - protein, 4 aa, synthetic construct
HPQD

SEQ ID NO: 194 - protein, 4 aa, synthetic construct
HDLA

SEQ ID NO: 195 - protein, 4 aa, synthetic construct
IDIS

SEQ ID NO: 196-197 - skipped (2 entries)

SEQ ID NO: 198 - protein, 4 aa, synthetic construct
ITNF

SEQ ID NO: 199 - protein, 4 aa, synthetic construct
LGSP

SEQ ID NO: 200 - protein, 4 aa, synthetic construct
LQKI

SEQ ID NO: 201 - protein, 4 aa, synthetic construct
VQNI

SEQ ID NO: 202 - protein, 4 aa, synthetic construct
VNAK

SEQ ID NO: 203-205 - skipped (3 entries)

SEQ ID NO: 206 - protein, 4 aa, synthetic construct
YENI

SEQ ID NO: 207 - protein, 7 aa, synthetic construct
  REGION 1..7: note = Description of sequence: Kinetic constant, table 1 peptide: 1
PCGGKAY

SEQ ID NO: 208 - protein, 7 aa, synthetic construct
  REGION 1..7: note = Description of sequence: Kinetic constant, table 1 peptide: 2
PCGSSKY

SEQ ID NO: 209 - protein, 7 aa, synthetic construct
  REGION 1..7: note = Description of sequence: Kinetic constant, table 1 peptide: 3
PCASKAY

SEQ ID NO: 210 - protein, 7 aa, synthetic construct
  REGION 1..7: note = Description of sequence: Kinetic constant, table 1 peptide: 4
PCSHKGY

SEQ ID NO: 211 - protein, 7 aa, synthetic construct
  REGION 1..7: note = Description of sequence: Kinetic constant, table 1 peptide: 5
PCEFTRY

SEQ ID NO: 212 - protein, 7 aa, synthetic construct
  REGION 1..7: note = Description of sequence: Kinetic constant, table 1 peptide: 6
PCAHKAY

SEQ ID NO: 213 - protein, 7 aa, synthetic construct
  REGION 1..7: note = Description of sequence: Kinetic constant, table 1 peptide: 7
PCGHKKY

SEQ ID NO: 214 - protein, 7 aa, synthetic construct
  REGION 1..7: note = Description of sequence: Kinetic constant, table 1 peptide: 8
PCGHKPY

SEQ ID NO: 215 - protein, 7 aa, synthetic construct
  REGION 1..7: note = Description of sequence: Kinetic constant, table 1 peptide: 9
PCGHKAY

SEQ ID NO: 216 - protein, 7 aa, synthetic construct
  REGION 1..7: note = Description of sequence: Kinetic constant, table 1 peptide: 10
PCAHKPY

SEQ ID NO: 217 - protein, 7 aa, synthetic construct
  REGION 1..7: note = Description of sequence: Kinetic constant, table 1 peptide: 11
PCHDETY

SEQ ID NO: 218 - protein, 6 aa, synthetic construct
  REGION 1..6: note = Description of sequence: Motif
PCGHKP

SEQ ID NO: 219 - protein, 15 aa, synthetic construct
  REGION 1..15: note = Description of sequence: Protein substrate
CGHKPGSSGSSRBDV

SEQ ID NO: 220 - protein, 15 aa, synthetic construct
  REGION 1..15: note = Description of sequence: Protein substrate
  MOD_RES <1: note = Cysteine bound to expression vector
CGHKPGSSGSSRBDV

SEQ ID NO: 221 - protein, 4 aa, synthetic construct
  REGION 1..4: note = Description of sequence: Protein substrate
  MOD_RES <1: note = Arginine bound to expression vector
RBDV

SEQ ID NO: 222 - protein, 6 aa, synthetic construct
  REGION 1..6: note = Description of sequence: Cleavage site
ENLYFQ

SEQ ID NO: 223 - protein, 5 aa, synthetic construct
  REGION 1..5: note = Description of sequence: Recognition motif
CGHKP

SEQ ID NO: 224 - protein, 11 aa, synthetic construct
  REGION 1..11: note = Description of sequence: Recognition motif
CGHKPGSSGSS

SEQ ID NO: 225 - protein, 11 aa, synthetic construct
  REGION 1..11: note = Description of sequence: Peptide library motif
  VARIANT 3..6: note = alanine, arginine, asparagine, aspartic acid, glutamine, glutamic acid, glycine, histidine, isoleucine, leucine, lysine, phenylalanine, proline, serine, threonine, tryptophan, tyrosine, valine, Nle, bAla and Abu
PCXXXXAARRM

SEQ ID NO: 226 - protein, 10 aa, synthetic construct
  REGION 1..10: note = Description of sequence: Peptide library motif
  VARIANT 2..5: note = alanine, arginine, asparagine, aspartic acid, glutamine, glutamic acid, glycine, histidine, isoleucine, leucine, lysine, phenylalanine, proline, serine, threonine, tryptophan, tyrosine, valine, Nle, bAla and Abu
CXXXXAARRM

SEQ ID NO: 227 - protein, 10 aa, synthetic construct
  REGION 1..10: note = Description of sequence: Peptide library motif
  MOD_RES 1: note = Cysteine bound to Dabcyl-CBT adduct
  VARIANT 2..5: note = alanine, arginine, asparagine, aspartic acid, glutamine, glutamic acid, glycine, histidine, isoleucine, leucine, lysine, phenylalanine, proline, serine, threonine, tryptophan, tyrosine, valine, Nle, bAla and Abu
CXXXXAARRM

SEQ ID NO: 228 - protein, 6 aa, synthetic construct
  REGION 1..6: note = Description of sequence: Kinetic constant
CGHKPY

SEQ ID NO: 229 - protein, 6 aa, synthetic construct
  REGION 1..6: note = Description of sequence: Kinetic constant
  MOD_RES 1: note = Cysteine bound to 6-Carboxyfluorescein
CGHKPY

SEQ ID NO: 230 - protein, 5 aa, synthetic construct
  REGION 1..5: note = Description of sequence: Resin bound linker
  MOD_RES 1: note = Methionine bound to 6-Carboxyfluorescein
  MOD_RES 4: note = bAla
  MOD_RES 5: note = bAla
MRRAA

SEQ ID NO: 231 - protein, 445 aa, Aeromonas veronii
MGSSHHHHHHSSGLVPRGSHMSSPLHYVLDGIHCEPHFFTVPLDHQQPDDEETITLFGRTLCRKDRLDDELPWLLYLQGGPGFGAPRPSANGGWIKRALQEFRVLLLDQRGTGHSTPIHAELLAHLNPRQQADYLSHFRADSIVRDAELIREQLSPDHPWSLLGQSFGGFCSLTYLSLFPDSLHEVYLTGGVAPIGRSADEVYRATYQRVADKNRAFFARFPHAQAIANRLATHLQRHDVRLPNGQRLTVEQLQQQGLDLGASGAFEELYYLLEDAFIGEKLNPAFLYQVQAMQPFNTNPVFAILHELIYCEGAASHWAAERVRGEFPALAWAQGKDFAFTGEMIFPWMFEQFRELIPLKEAAHLLAEKADWGPLYDPVQLARNKVPVACAVYAEDMYVEFDYSRETLKGLSNSRAWITNEYEHNGLRVDGEQILDRLIRLNRDC

SEQ ID NO: 232 - protein, 119 aa, synthetic construct
  REGION 1..119: note = Description of sequence: cloning construct component
MPCKTSNTIRVLLPNQEWTVVKVRNGMSLHDSLMKALKRHGLQPESSAVFRLLHEHKGKKARLDWNTDAASLIGEELQVDFLDHVPLTTHNFARKTFLKLGIHRDGSSGSSLEHHHHHH

SEQ ID NO: 233 - protein, 123 aa, synthetic construct; identical to SEQ ID NO: 90
  REGION 1..123: note = Description of sequence: cloning construct component

SEQ ID NO: 234 - protein, 129 aa, synthetic construct
  REGION 1..129: note = Description of sequence: cloning construct component
MPCGHKPGSSGSSKTSNTIRVLLPNQEWTVVKVRNGMSLHDSLMKALKRHGLQPESSAVFRLLHEHKGKKARLDWNTDAASLIGEELQVDFLDHVPLTTHNFARKTFLKLGIHRDGSSGSSLEHHHHHH

(19) United States
(12) Patent Application Publication
(10) Pub. No.: US 2026/0337656 A1
(43) Pub. Date: Oct. 29, 2026

(54) METHODS OF SYNTHESIZING PEPTIDES WITH FREE N-TERMINAL CYSTEINE AND PRODUCTS THEREOF

(71) Applicant: OHIO STATE INNOVATION FOUNDATION, Columbus, OH (US)

(72) Inventor: Dehua PEI, Columbus, OH (US)

(21) Appl. No.: 19/105,522

(22) PCT Filed: Aug. 24, 2023
(86) PCT No.: PCT/US2023/072809

§ 371 Date: Feb. 21, 2025

Related U.S. Application Data
(60) Provisional application No. 63/400,534, filed on Aug. 24, 2022.

(30) Foreign Application Priority Data
Aug. 24, 2023 (US) 63400534

(51) Int. Cl. C12P 21/06; C12N 15/70

(52) U.S. Cl. CPC C12P 21/06; C12N 15/70

(57) ABSTRACT

This disclosure describes methods of site-specific modification of peptides for conjugation, and more particularly methods for synthesizing peptides with N-terminal cysteine and uses of the same.

### CROSS-REFERENCE TO RELATED APPLICATIONS

This application claims the benefit of priority to U.S. Provisional Application No. 63/400,534 filed Aug. 24, 2022, the disclosure of which is incorporated herein by reference in its entirety.

### STATEMENT REGARDING FEDERALLY SPONSORED RESEARCH OR DEVELOPMENT

This invention was made with government support under grant/contract number GM122459 awarded by the National Institutes of Health. The government has certain rights in the invention.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Aug. 22, 2023, is entitled “103361-337WO1_ST26.xml”, and is 267,728 bytes in size.

### TECHNICAL FIELD

This disclosure relates to methods of site-specific modification of peptides for conjugation, and more particularly to methods for synthesizing peptides with N-terminal cysteine and uses of the same.

### BACKGROUND

Site-specific modification of proteins is of great utility in fundamental research as well as biotechnology. Common applications include fluorescent labeling of proteins for biochemical/biophysical characterization or in vivo imaging, immobilization of proteins to polymers or surfaces, antibody-drug conjugates, and PEGylation of proteins for improved pharmacokinetic properties. However, despite decades of efforts by many investigators,^{1,2} an efficient, universal method for site-specific protein conjugation is not yet available, due to vast heterogeneity in the physicochemical properties of different proteins.

A popular approach involves the modification of proteins at their N-termini, as the N-terminus is usually solvent exposed, and modification at the N-terminus is less likely to adversely affect the folding and/or function of the protein. In addition, a variety of strategies have been developed to modify N-terminal amino acids directly or convert them into unique functional groups for further ligations.³ One approach takes advantage of the unique properties of an N-terminal cysteine, which selectively reacts with aldehydes to form thiazolidines,⁴ thioesters to form amides (i.e., native chemical ligation⁵), or 2-cyanobenzothiazole (CBT) to form 2-thiazolines.⁶ This approach, of course, relies on the effective production of proteins with a free N-terminal cysteine.

Several methods have been developed to produce proteins with N-terminal cysteines, which rarely occur naturally. In principle, the simplest method is to append a Met-Cys dipeptide to the N-terminus of a protein of interest and express the modified protein in bacteria (e.g., Escherichia coli).⁷ The N-terminal formyl-Met moiety is removed co-translationally by the sequential action of peptide deformylase (PDF) and methionine aminopeptidase (MetAP) to produce an N-terminal cysteine.⁸ Unfortunately, this method is complicated by further reaction of the N-terminal cysteine with intracellular aldehyde/ketone metabolites (e.g., pyruvate) and sequestration of the N-terminal cysteine in the form of thiazolidine derivatives.⁷ Moreover, this method is not compatible with protein expression in eukaryotic cells, in which most proteins are N-terminally acetylated.⁹ To overcome the above limitations, Hauser and Ryan fused a leader peptide (pelB) to the N-termini of proteins, resulting in the export of the fusion proteins into the periplasmic space; subsequent processing by leader peptidase(s) in the periplasmic space leaves an N-terminal cysteine.¹⁰ However, this method is only applicable to proteins that can be exported into the periplasmic space and often results in poor protein expression yields.^{11,12} Currently, the most common strategy involves the expression of fusion proteins containing the recognition sequence of a sequence-selective protease [e.g., tobacco etch virus (TEV),¹³ thrombin,¹⁴ and factor Xa¹⁴], which is selectively cleaved by the protease after the recognition sequence to leave an N-terminal cysteine. The main challenge of the latter method is that these proteases are not completely sequence specific and frequently cleave a protein of interest at unintended secondary recognition sites.¹⁴ Further, the cleavage at the intended site (ENLYFQ) (SEQ ID NO: 222) by TEV can be extremely slow (relative to nonspecific cleavage at secondary sites), likely because the TEV recognition site is sterically blocked owing to its interaction with the protein surface (unpublished results).

There is a clear need for alternative methods that efficiently, reliably, and cost-effectively produce proteins with free N-terminal cysteines.

### SUMMARY

The present disclosure provides methods for selectively preparing peptides having an N-terminal cysteine residue that avoid reaction with aldehyde/ketone residues within the cell by protecting the N-terminal cysteine with a proline residue. The peptide having an N-terminal proline residue is then selectively cleaved ex vivo to provide the N-terminal cysteine-containing peptide. These methods may be adapted for use in both prokaryotic and eukaryotic expression systems and avoid cleavage at secondary sites which may occur with the alternative protease systems available.

In one aspect, a method is provided of preparing a peptide having an N-terminal cysteine residue of Formula I:

Cys-[peptide] (I)
wherein Cys is the N-terminal cysteine residue and [peptide] comprises an amino acid sequence having at least five residues;
the method comprising:
(i) expressing within a prokaryotic cell a first peptide of Formula II-a:

fMet-Pro-Cys-[peptide] (II-a)
fMet is an N-formylmethionine residue and Pro is a proline residue, and
wherein the prokaryotic cell further expresses a peptide deformylase (PDF) and a methionine amino peptidase (MetAP) such that the fMet residue is cleaved in situ to provide a second peptide of Formula III:

Pro-Cys-[peptide] (III);
(ii) isolating the second peptide from the prokaryotic cell;
(iii) contacting the second peptide of Formula III with a prolyl aminopeptidase (ProAP) to provide the peptide of Formula I.

Also provided is a method of preparing a peptide of Formula I:

Cys-[peptide] (I)
wherein Cys comprises a cysteine residue at the N-terminus and [peptide] comprises a peptide sequence having at least five amino acid residues;
the method comprising:
(i) expressing within a eukaryotic cell a first peptide of Formula II-b:

Met-Pro-Cys-[peptide] (II-b)
wherein Met comprises a methionine residue and Pro comprises a proline residue,
wherein the cell further expresses a methionine amino peptidase (MetAP) such that the Met residue is cleaved in situ to provide a second peptide of Formula III:

Pro-Cys-[peptide] (III);
(ii) isolating the second peptide from the eukaryotic cell;
(iii) contacting the second peptide of Formula III with a prolyl aminopeptidase to provide the peptide of Formula I.

A peptide of Formula I is also provided prepared by the methods described herein.

A method is also provided for preparing a chemically-modified peptide, the method comprising:
(i) preparing a peptide having an N-terminal cysteine residue according to the methods described herein; and
(ii) conjugating the peptide with a ligand, wherein the ligand comprises a moiety capable of reacting with the N-terminal cysteine residue.

A chemically-modified peptide is also provided prepared according to the methods described herein.

Further provided is a method for determining the specificity of a prolyl aminopeptidase (ProAP) comprising:
(i) providing a library of peptides, wherein each peptide has an N-terminal sequence comprising Pro-Cys, wherein Pro is an N-terminal proline and Cys is cysteine;
(ii) contacting the library of peptides with the prolyl aminopeptidase such that Pro is cleaved in a portion of the library of peptides to provide a cleaved portion;
(iii) contacting the library of peptides with a label having a moiety capable of reacting with an N-terminal cysteine residue such that the cleaved portion is labeled to provide a labeled portion;
(iv) isolating the labeled portion; and
(v) sequencing the labeled portion.

The details of one or more aspects of the disclosure are set forth in the accompanying drawings and the description below. Other features, objects, and advantages of the disclosure will be apparent from the description and drawings, and from the claims.

### DESCRIPTION OF DRAWINGS

FIG. 1 provides a representative scheme showing the generation and selective modification of an N-terminal cysteine in proteins. PDF, peptide deformylase; MetAP, methionine aminopeptidase; ProAP, prolyl aminopeptidase; FAM, fluorescein.

FIGS. 2A-2C show the determination of A. sobria ProAP substrate specificity by peptide library screening. (FIG. 2A) Reactions involved in peptide library screening (SEQ ID NOs: 225-227, left to right, top to bottom, respectively). (FIG. 2B) Photograph of a portion of the library after treatment with ProAP (1 μM) and Dabcyl-CBT. (FIG. 2C) Histogram showing the sequence specificity of A. sobria ProAP at the P2′-P5′ positions, as displayed by the number of selected sequences (y axis) containing a given amino acid (x axis) at a particular position (z axis).

FIGS. 3A-3B show N-Terminal labeling of peptide 8 with fluorescein (FIG. 3A) Reactions mediated by ProAP and native chemical ligation (SEQ ID NOs: 214, 228, 229, top to bottom respectively). (FIG. 3B) UPLC-MS analysis of peptide 8 and its reaction products (monitored at 280 nm).

FIGS. 4A-4B show efficiency of proline removal from RBDV proteins (SEQ ID NO: 87) by ProAP. (FIG. 4A) Representative MALDI-FT-ICR mass spectra of PCGHKP-RBDV (25 μM) (SEQ ID NO: 95) after treatment with ProAP (1 μM) for 0, 60, and 480 min. Ions observed correspond to the [M+2H]²⁺ species. (FIG. 4B) Plot of the percentage of proline removal from PCGHKP-RBDV (SEQ ID NO: 95) and PCGHKP-(GSS)₂-RBDV (SEQ ID NO: 96) as a function of reaction time.

FIGS. 5A-5C show conjugation of fluorescein and CPP12 to the N-terminus of RBDV. (FIG. 5A) MALDI-FT-ICR mass spectra of PCGHKP-(GSS)₂-RBDV (SEQ ID NO: 96) before any treatment (calculated m/z for [M+2H]²⁺ 7157.21; observed 7157.19), after treatment with ProAP (calculated m/z for [M+2H]²⁺ 7108.68; observed 7108.65), and after treatment with ProAP and FAM-CO-SR (calculated for [M+2H]²⁺ 7288.21; observed 7288.16) or CPP12-CBT (calculated m/z for [M+2H]²⁺ 7988.11; observed 7988.15). (FIG. SB) SDS-PAGE analysis of PCGHKP-(GSS)₂-RBDV (SEQ ID NO: 96) before and after treatment with ProAP and/or FAM-CO-SR. Top panel, FAM fluorescence upon irradiation of the gel with a UV lamp; lower panel, the same gel stained with Coomassie blue. (FIG. 5C) Coomassie blue-stained SDS-PAGE gel showing the modification of PCGHKP-(GSS)₂-RBDV (SEQ ID NO: 96) by ProAP and/or CPP12-CBT.

FIG. 6 provides a scheme showing the synthesis of resin-bound ProAP substrate peptide library. HATU, (1-[bis(dimethylamino)methylene]-1H-1,2,3-triazolo[4,5-b]pyridinium 3-oxide hexafluorophosphate; DIPEA, diisopropylethylamine (SEQ ID NO: 230).

FIGS. 7A-7M provides the structures, preparative HPLC chromatograms, UPLC chromatograms, and mass spectra of compounds used in Example 1. HRMS analysis was performed by MALDI FT-ICR mass spectrometry SEQ ID NOs: 207-217, FIG. 7B-7L, respectively.

FIGS. 8A-8B provides the plasmids used for bacterial expression of ProAP and RBDV in Example 1. (FIG. 8A) Restriction map of plasmid pET-15b(+)-His₆-ProAP. The plasmids for RBDV expression have a similar restriction map. (FIG. 8B) Amino acid sequences for A. sobria ProAP and RBDV proteins (SEQ ID NOs: 231-234).

Like reference symbols in the various drawings indicate like elements.

### DETAILED DESCRIPTION

The following description of the disclosure is provided as an enabling teaching of the disclosure in its best, currently known aspects. Many modifications and other aspects disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual aspects described and illustrated herein has discrete components and features which may be readily separated from or combined with the features of any of the other several aspects without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a label”, “an amino acid”, or “a cell”, includes, but is not limited to, two or more such labels, amino acids, or cells, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “culture”, “cultivate”, and “ferment” are used interchangeably and refer to the intentional growth, propagation, proliferation, and/or enablement of metabolism, catabolism, and/or anabolism of one or more cells. The combination of both growth and propagation may be termed proliferation. Culture does not refer to the growth or propagation of cells in nature or otherwise without human intervention.

The term “growth” means an increase in cell size, total cellular contents, and/or cell mass or weight of a cell.

The term “propagation” refers to an increase in cell number via cell division.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which are also commonly referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, and fusion proteins, among others. The polypeptides may include natural peptides, recombinant peptides, synthetic peptide, or a combination thereof.

“Nucleic acid” or “oligonucleotide” or “polynucleotide” or grammatical equivalents used herein means at least two nucleotides covalently linked together. The term “nucleic acid” includes single-, double-, or multiple-stranded DNA, RNA and analogs (derivatives) thereof. Oligonucleotides are typically from about 5, 6, 7, 8, 9, 10, 12, 15, 25, 30, 40, 50 or more nucleotides in length, up to about 100 nucleotides in length. Nucleic acids and polynucleotides are polymers of any length, including longer lengths, e.g., 200, 300, 500, 1000, 2000, 3000, 5000, 7000, 10,000, etc. In certain aspects, the nucleic acids herein contain phosphodiester bonds. In other aspects, nucleic acid analogs are included that may have alternate backbones. The term encompasses nucleic acids containing known analogues of natural nucleotides which have similar or improved binding properties, for the purposes desired, as the reference nucleic acid. A particular nucleic acid sequence also encompasses “splice variants.” Similarly, a particular protein encoded by a nucleic acid encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of splice variants is discussed in Leicher, et al., J. Biol. Chem. 273(52):35095-35101 (1998).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, RNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listing, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The nucleotide sequence that encodes a protein or an RNA may also include introns to the extent that the nucleotide sequence encoding the protein may in some versions contain an intron(s).

The term “expression” as used herein is defined as the transcription and/or translation of a particular nucleotide sequence driven by its promoter.

The term “promoter” or “regulatory element” refers to a region or sequence determinants located upstream or downstream from the start of transcription and which are involved in recognition and binding of RNA polymerase and other proteins to initiate transcription. Promoters need not be of origin in the cell used, for example, promoters derived from viruses or from other organisms can be used in the compositions or methods described herein.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In some aspects, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide).

The term “expression cassette” refers to a nucleic acid construct, which when introduced into a host cell, results in transcription and/or translation of a RNA or polypeptide, respectively. In some aspects, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some aspects, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some aspects, the expression cassette comprises a promoter operably linked to a second nucleic acid (e.g. polynucleotide) and a terminator operably linked to the second nucleic acid (e.g. polynucleotide) as the result of human manipulation. In some aspects, the expression cassette comprises an endogenous promoter. In some aspects, the expression cassette comprises an endogenous terminator. In some aspects, the expression cassette comprises a synthetic (or non-natural) promoter. In some aspects, the expression cassette comprises a synthetic (or non-natural) terminator.

The term “transfected” or “transformed” or “transduced” as used herein refers to a process by which exogenous nucleic acid is transferred or introduced into a host cell. A “transfected” or “transformed” or “transduced” cell is one which has been transfected, transformed, or transduced with exogenous nucleic acid.

A “vector” is a composition of matter which comprises an isolated nucleic acid and which can be used to deliver the isolated nucleic acid to the interior of a cell. Numerous vector are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of a nucleic acid into cells, such as, for example, polylysine compounds, liposomes, and the like. Examples of viral vectors include, but are not limited to, adenoviral vectors, adeno-associated virus vectors, retroviral vectors, and the like.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.nebi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

Methods for synthesizing sequences and bringing sequences together are well established and known to those of skill in the art. For example, in vitro mutagenesis and selection, site-directed mutagenesis, error prone PCR (Melnikov et al., Nucleic Acids Research, 27(4):1056-1062 (Feb. 15, 1999)), “gene shuffling” or other means can be employed to obtain mutations of naturally occurring genes.

The present disclosure provides methods for selectively preparing peptides having an N-terminal cysteine residue that avoid reaction with aldehyde/ketone residues within the cell by protecting the N-terminal cysteine with a proline residue. The peptide having an N-terminal proline residue is then selectively cleaved ex vivo to provide the N-terminal cysteine containing peptide. These methods may be adapted for use in both prokaryotic and eukaryotic expressions systems and avoids cleavage at secondary sites which may occur with protease systems also used.

Thus, in one aspect, a method is provided of preparing a peptide having an N-terminal cysteine residue of Formula I:

Cys-[peptide] (I)
wherein Cys is the N-terminal cysteine residue and [peptide] comprises an amino acid sequence having at least five residues;
the method comprising:
(i) expressing within a prokaryotic cell a first peptide of Formula II-a:

fMet-Pro-Cys-[peptide] (II-a)
wherein fMet is an N-formylmethionine residue and Pro is a proline residue, and
wherein the prokaryotic cell further expresses a peptide deformylase (PDF) and a methionine amino peptidase (MetAP) such that the fMet residue is cleaved in situ to provide a second peptide of Formula III:

Pro-Cys-[peptide] (III);
(ii) isolating the second peptide from the prokaryotic cell;
iii) contacting the second peptide of Formula III with a prolyl aminopeptidase (ProAP) to provide the peptide of Formula I.
[peptide] may comprise any amino acid (i.e., peptide) sequence of interest. In some aspects, [peptide] may comprise a short chain peptide or an oligopeptide. In other aspects, [peptide] may comprise a protein. In some aspects, [peptide] may comprise a natural peptide, a recombinant peptide, a synthetic peptide, or any combination of the same (e.g., a fusion peptide).

The prokaryotic cell may comprise any cell from a suitable prokaryotic organism. Representative examples of prokaryotic cells which may be used include, but are not limited to, an Escherichia coli cell or a Bacillus subtilis cell.

The prokaryotic cell further expresses a peptide deformylase (PDF) and a methionine amino peptidase (MetAP). The PDF, the MetAP, or both may be endogenous to the prokaryotic cell or may be exogenously derived by expression from an expression vector.

In another aspect, a method is provided of preparing a peptide of Formula I:

Cys-[peptide] (I)
wherein Cys comprises a cysteine residue at the N-terminus and [peptide] comprises a peptide sequence having at least five amino acid residues;
the method comprising:
(i) expressing within a eukaryotic cell a first peptide of Formula II-b:

Met-Pro-Cys-[peptide] (II-b)
wherein Met comprises a methionine residue and Pro comprises a proline residue,
wherein the cell further expresses a methionine amino peptidase (MetAP) such that the Met residue is cleaved in situ to provide a second peptide of Formula III:

Pro-Cys-[peptide] (III);
(ii) isolating the second peptide from the eukaryotic cell;
(iii) contacting the second peptide of Formula III with a prolyl aminopeptidase to provide the peptide of Formula I.

The eukaryotic cell may comprise any cell from a suitable eukaryotic organism. Representative examples include, but are not limited to, Pichia pastoris, Kluyveromyces lactis, Saccharomyces cerevisiae, and mammalian cells (such as Chinese hamster ovary (CHO), COS, HEK, and HeLa).

The eukaryotic cell further expresses a methionine amino peptidase (MetAP). The MetAP may be endogenous to the eukaryotic cell or may be exogenously derived by expression from an expression vector.

In some aspects, it may be preferred to select a ProAP which has good activity for longer chain peptides, e.g., proteins. Representative examples of such ProAPs include, but are not limited to, a ProAP isolated from Aeromonas sobria, Aspergillus oryzae, Aspergillus niger, or Debaryomyces hansenii. In other aspects, it may be preferred to select a ProAP which has good activity selectively for shorter chain peptides, for example a ProAP isolated from Bacillus coagulans. In general, it is preferred to select a ProAP which shows high selectivity for an N-terminal proline residue and which does not cleave other N-terminal residues. Thus, a ProAP isolated from Streptomyces lavendulae, Grifola frondose, Phanerochaete chrysosporium, Neisseria gonorrhoeae, or Talaromyces emersonii may be less preferred in certain contexts due to its lower selectivity for N-terminal proline compared to other N-terminal residues. However, a person of ordinary skill may select any ProAP that may be sufficiently suitable for cleavage of the particular peptide of interest.

In some aspects, [peptide] further comprises a recognition sequence for the ProAP. The recognition sequence may facilitate binding of the ProAP to the second peptide of Formula III, thus facilitating cleavage of the N-terminal proline residue.

In some aspects, the recognition sequence may be selected from selected from:

(SEQ ID NO. 1)
GNRS;
(SEQ ID NO. 2)
GFKG;
(SEQ ID NO. 3)
AAGQ;
(SEQ ID NO. 4)
ALKH;
(SEQ ID NO. 5)
EFAR;
(SEQ ID NO. 6)
GHKF;
(SEQ ID NO. 7)
GGKU;
(SEQ ID NO. 8)
AAAR;
(SEQ ID NO. 9)
ALKN;
(SEQ ID NO. 10)
FGSV;
(SEQ ID NO. 11)
GHYA;
(SEQ ID NO. 12)
GFSF;
(SEQ ID NO. 13)
AURU;
(SEQ ID NO. 14)
AAKF;
(SEQ ID NO. 15)
RNYN;
(SEQ ID NO. 16)
GGKA;
(SEQ ID NO. 17)
AALK;
(SEQ ID NO. 18)
ASKA;
(SEQ ID NO. 19)
SFKG;
(SEQ ID NO. 20)
MIHE;
(SEQ ID NO. 21)
GGRA;
(SEQ ID NO. 22)
AHKA;
(SEQ ID NO. 23)
AFKN;
(SEQ ID NO. 24)
SHKG;
(SEQ ID NO. 25)
GNFK;
(SEQ ID NO. 26)
ALAR;
(SEQ ID NO. 27)
AGVR;
and
(SEQ ID NO. 28)
EFTR.

In some aspects, the recognition sequence is selected from:

(SEQ ID NO. 29)
GAGK;
(SEQ ID NO. 30)
GRAU;
(SEQ ID NO. 31)
GFHG;
(SEQ ID NO. 32)
AAGK;
(SEQ ID NO. 33)
PARV;
(SEQ ID NO. 34)
GYLS;
(SEQ ID NO. 35)
GHNK;
(SEQ ID NO. 36)
GQUY;
(SEQ ID NO. 37)
UAKS;
(SEQ ID NO. 38)
ERKG;
(SEQ ID NO. 39)
GAUR;
(SEQ ID NO. 40)
GYGI;
(SEQ ID NO. 41)
GAFI;
(SEQ ID NO. 42)
UUKN;
(SEQ ID NO. 43)
EIGR;
(SEQ ID NO. 44)
GYAF;
(SEQ ID NO. 45)
GNKY;
(SEQ ID NO. 46)
GNKG;
(SEQ ID NO. 47)
UASR;
(SEQ ID NO. 48)
RHTR;
(SEQ ID NO. 49)
GHFG;
(SEQ ID NO. 50)
GLKI;
(SEQ ID NO. 51)
GHAG;
(SEQ ID NO. 52)
UMKG;
(SEQ ID NO. 53)
RHFI;
(SEQ ID NO. 54)
GFGY;
(SEQ ID NO. 55)
GGRV;
(SEQ ID NO. 56)
GVYG;
(SEQ ID NO. 57)
SLKM;
(SEQ ID NO. 58)
RTNL;
(SEQ ID NO. 59)
GMLY;
(SEQ ID NO. 60)
GYUN;
(SEQ ID NO. 61)
GALK;
(SEQ ID NO. 62)
SLYP;
(SEQ ID NO. 63)
KPAK;
(SEQ ID NO. 64)
GSSK;
(SEQ ID NO. 65)
GFIG;
(SEQ ID NO. 66)
AHKK;
(SEQ ID NO. 67)
SUKG;
(SEQ ID NO. 68)
YEML;
(SEQ ID NO. 69)
GAIK;
(SEQ ID NO. 70)
GGKF;
(SEQ ID NO. 71)
AHNK;
(SEQ ID NO. 72)
SFNK;
(SEQ ID NO. 73)
YRDU;
(SEQ ID NO. 74)
GUGR;
(SEQ ID NO. 75)
GRAL;
(SEQ ID NO. 76)
AUKL;
(SEQ ID NO. 77)
SAKK;
(SEQ ID NO. 78)
FRGK;
(SEQ ID NO. 79)
GYSK;
(SEQ ID NO. 80)
GRGA;
(SEQ ID NO. 81)
AMIK;
(SEQ ID NO. 82)
SNKG;
(SEQ ID NO. 83)
LUFN;
and
(SEQ ID NO. 84)
GMGK.

In some aspects, the first peptide is expressed from at least one non-naturally occurring gene within the cell. The at least one non-naturally occurring gene may be integrated into the genome of the cell or may be integrated into an expression vector. In some aspects, the expression vector may comprise a plasmid or a virus. In some aspects, the expression vector comprises a plasmid. In other aspects, the expression vector may comprise a virus, for example an adenoviral vector, an adeno-associated virus vectors, or a retroviral vector.

In some aspects, the at least one non-naturally occurring gene may be operably linked to a promoter within the expression vector. A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In aspects, a promoter is operably linked with a coding sequence when it is capable of affecting (e.g. modulating relative to the absence of the promoter) the expression of a protein from that coding sequence (i.e., the coding sequence is under the transcriptional control of the promoter). In some aspects, the promoter may be constitutively active or inducibly active. Examples of suitable prokaryotic expression vectors include, but are not limited to, the pGEX series (using the TAC promoter) and pET series (using the T7 promoter) of expression vectors. Examples of suitable eukaryotic expression vectors include, but are not limited to, adenoviral vectors, pSV (using the SV40 promoter) and pCMV (using the CMV promoter) series of plasmic vectors, vaccinia and retroviral vectors, and the elongation factor (EF)-1 promoter.

In another aspect, a peptide of Formula I is provided prepared by the methods described herein.

A method is also provided for preparing a chemically-modified peptide, the method comprising:
(i) preparing a peptide having an N-terminal cysteine residue according to the methods described herein; and
(ii) conjugating the peptide with a ligand, wherein the ligand comprises a moiety capable of reacting with the N-terminal cysteine residue.

The ligand may comprise a label, a drug, a protein, an antibody, a nucleic acid, a lipid, a saccharide, a polymer, a nanomaterial, a cell-penetrating peptide, a linear or cyclic peptide, an imaging agent, a theranostic, a radionuclide, a targeting agent, or combinations thereof.

In some aspects, the ligand comprises a label, including biotins, bioconjugating groups, a fluorescent marker (e.g., fluorophores), radiomarkers, organocatalysts, dye, quantum dot, enzyme, enzyme substrate, and other detectable markers), metal catalyst, small molecule ligands, drugs, PROTAC® (E3 ligase ligand), or LYTAC® (cation-independent mannose-6-phosphate receptor (CI-M6PR) ligand) or small molecule inhibitors). A label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorophores) and enzymes or chemiluminescent markers that react with colorimetric substrates (e.g., horseradish peroxidase).

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA, Aequorin (Photoprotein); AFPs-AutoFluorescent Protein-(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR, Bodipy TR ATP; Bodipy TR-X SE; BO-PRO™-1; BO-PRO™-3; Brilliant Sulphoflavin FF; BTC; BTC-SN; Calcein; Calcein Blue; Calcium Crimson-; Calcium Green; Calcium Green-1 Ca²⁺ Dye; Calcium Green-2 Ca²⁺, Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DIR; DIR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH), Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type′ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DID); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Monobromobimane; Red; Mitramycin; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARFI; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20, SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1, YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

In some aspects, the ligand may comprise a therapeutic agent. The term “therapeutic agent” includes any synthetic or naturally occurring biologically active compound or composition of matter which, when administered to an organism (either human or a nonhuman animal), induces a desired pharmacologic, immunogenic, and/or physiologic effect by local and/or systemic action. The term therefore encompasses those compounds or chemicals traditionally regard as drugs, vaccines, and biopharmaceuticals including molecules such as proteins, peptides, hormones, nucleic acids, gene constructs and the like. Examples of therapeutic agents are described in well-known literature references such as the Merk Index (14^{th} Edition), the Physician's Desk Reference (64^{th} Edition), and The Pharmacological Basis of Therapeutics (12^{th} Edition), and they include, without limitation, medicaments; vitamins; mineral supplements, substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiandrenergics, antiarrhythmics, antihypertensive agents, hormones, and nutrients, antiarthritics, antiasthmatic agents, anticonvulsants, antihistamines, antinauscants, antineoplastics, antipruritics, antipyretics, antispasmodics, cardiovascular preparations (including calcium channel blockers, beta blockers, and beta-agonists), antihypertensives, diuretics, vasodilators, central nervous system stimulants, cough and cold preparations, decongestants, diagnostics, bone growth stimulants and bone resorption inhibitors, immunosuppressives, muscle relaxants, psychostimulants, sedatives, tranquilizers, proteins, peptides, and fragments thereof (whether naturally occurring, chemically synthesized or recombinantly produced), and nucleic acid molecules (polymeric forms of two or more nucleotides, either ribonucleotides (RNA) or deoxyribonucleotides (DNA) including both double and single-stranded molecules, gene constructs, expression vectors, antisense molecules and the like), small molecules and other biologically active macromolecules such as, for examples, proteins and enzymes. The agent may be a biologically active agent used in medical, including veterinary, applications and in agriculture, such as with plants, as well as other areas.

In some aspects, the moiety capable of reacting with the N-terminal cysteine residue comprises an aldehyde moiety, a ketone moiety, a thioester moiety, a 2-cyanobenzothiazolyl moiety, a cyclopropenone moiety, a 2-benzylacrylaldehyde moiety, or a 2-((alkylthio) (aryl)methylene) malononitrile moiety. Further details about such moieties suitable for reaction with an N-terminal cysteine residue are described in: Zhang, L.; Tam, J. P. Anal. Biochem. 1996, 233, 87-93; Dawson, P. E.; Muir, T. W.; Clark-Lewis, I.; Kent, S. B. H. Science 1994, 266, 776-779; Ren, H.; Xiao, F.; Zhan, K.; Kim, Y. P.; Xie, H.; Xia, Z.; Rao, J. Angew. Chem. Int. Ed. Engl. 2009, 48, 9658-9662; Alena Istrate, Michael B. Geeson, Claudio D. Navo, Barbara B. Sousa, Marta C. Marques, Ross J. Taylor, Toby Journeaux, Sebastian R. Oehler, Michael R. Mortensen, Michael J. Deery, Andrew D. Bond, Francisco Corzana, Gonzalo Jiménez-Osés, and Gonçalo J. L. Bernardes, Journal of the American Chemical Society 2022, 144, 23, 10396-10406; Yaqi Wu, Cong Li, Shihui Fan, Yibing Zhao, and Chuanliu Wu, Bioconjugate Chemistry 2021, 32, 9, 2065-2072; and Xiaoli Zheng, Zhuoru Li, Wei Gao, Xiaoting Meng, Xuefei Li, Louis Y. P. Luk, Yibing Zhao, Yu-Hsuan Tsai, and Chuanliu Wu, Journal of the American Chemical Society 2020, 142, 11, 5097-5103.

In another aspect, a chemically-modified peptide is also provided prepared according to the methods described herein.

In a further aspect, a method is provided for determining the specificity of a prolyl aminopeptidase comprising:
(i) providing a library of peptides, wherein each peptide has an N-terminal sequence comprising Pro-Cys, wherein Pro is an N-terminal proline and Cys is cysteine;
(ii) contacting the library of peptides with the prolyl aminopeptidase such that Pro is cleaved in a portion of the library of peptides to provide a cleaved portion;
(iii) contacting the library of peptides with a label having a moiety capable of reacting with an N-terminal cysteine residue such that the cleaved portion is labeled to provide a labeled portion;
(iv) isolating the labeled portion; and
(v) sequencing the labeled portion.

In some aspects, each peptide within the library of peptides is bound to a substrate. In some aspects, the substrate comprises a bead.

In some aspects, isolating the labeled portion comprises releasing the labeled portion from the substrate.

A number of aspects of the disclosure have been described. Nevertheless, it will be understood that various modifications may be made without departing from the spirit and scope of the invention. Accordingly, other aspects are within the scope of the following claims.

By way of non-limiting illustration, examples of certain aspects of the present disclosure are given below.

### EXAMPLES

The following examples are put forth so as to provide those of ordinary skill in the art with a complete disclosure and description of how the compounds, compositions, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

### Example 1. Generation of Proteins with Free N-Terminal Cysteine by Aminopeptidases

Efficient, site-specific, and bio-orthogonal conjugation of chemical functionalities to proteins is of great utility in fundamental research as well as industrial processes (e.g., the production of antibody-drug conjugates and immobilization of enzymes for biocatalysis). A popular approach involves reacting a free N-terminal cysteine with a variety of electrophilic reagents. However, current methods for generating proteins with N-terminal cysteines have significant limitations. In this example, we describe a method for producing recombinant proteins with free N-terminal cysteines by genetically fusing a Met-Pro-Cys sequence to the N-terminus of a protein of interest and subjecting the recombinant protein to the sequential action of methionine and proline aminopeptidases. The resulting protein was site-specifically labeled at the N-terminus with fluorescein and a cyclic cell-penetrating peptide through native chemical ligation and a 2-cyanobenzothiazole moiety, respectively. In addition, the optimal recognition sequence of Aeromonas sobria proline aminopeptidase was determined by screening a combinatorial peptide library and incorporated into the N-terminus of a protein of interest for the most efficient N-terminal processing.

### Experimental

### Materials

Reagents for peptide synthesis were obtained from Chem-Impex (Wood Dale, IL). All solvents and other chemical reagents were obtained from Sigma-Aldrich, Fisher Scientific (Pittsburgh, PA), or VWR (West Chester, PA) and used without further purification. Dithiothreitol (DTT), isopropyl β-D-1-thiogalactopyranoside (IPTG), protease inhibitor cocktail, chicken egg lysozyme, imidazole, and ampicillin were purchased from Sigma-Aldrich (St. Louis, MO).

### MALDI FT-ICR MS Analysis

Samples were analyzed using a Bruker Daltonics (Bremen, Germany) 15T Solarix Fourier transform-ion cyclotron resonance (FT-ICR) mass spectrometer equipped with a SmartBeam II frequency-tripled (355 nm) Nd:YAG laser utilizing a matrix-assisted laser desorption and ionization (MALDI) source. Samples were analyzed by dried drop utilizing CHCA matrix (5 mg/mL in 50% LC-MS grade acetonitrile with 0.1% trifluoroacetic acid) spotted at 2 μL on a stainless steel MALDI plate (3 replicates for each sample) unless otherwise noted. All samples were analyzed in positive ion mode over a mass range of m/z 300-10,000 using a 4M word time-domain dataset for high-resolution analysis. An average of 60 scans were taken with each scan averaging signal from 500 laser shots. The laser spot size was set to a small focus at a frequency of 2,000 Hz. External calibration was completed using a peptide calibration standard supplemented with insulin in CHCA matrix (Bruker Daltonics, Billerica, MA).

### Synthesis of Dabcyl-CBT

2-Cyano-6-aminobenzothiazole (18 mg, 0.1 mmol) was added to a mixture of Boc-glycine (88 mg, 0.5 mmol), N,N′-diisopropylcarbodiimide (85 μL, 0.55 mmol) and 4-dimethylaminopyridine (1.22 mg, 0.01 mmol) in DMF (600 μL). The mixture was stirred overnight at room temperature. The solution was transferred into a separatory funnel and ethyl acetate (10 mL) was added. The solution was extracted with 4 mL of water and 10 mL of brine. The organic phase was dried with Na₂SO₄ and evaporated. The crude product was redissolved in hexane and purified by silica gel column chromatography and using 1:4 ethyl acetate/hexanes as eluant. Evaporation of the solvent gave the pure product Boc-Gly-CBT (27 mg, 79% yield) as a white solid. 1H NMR (400 Hz, CDCl₃) ¿=1.48 (s, 9H), 3.96 (d, J=6.0 Hz, 2H), 5.20-5.40 (bs, 1H), 7.40 (d, J=9.0 Hz, 1H), 8.07 (d, J=9.0 Hz, 1H), 8.60 (s, 1H), 8.80-9.00 (bs, 1H).

Boc-Gly-CBT was treated with 20% trifluoroacetic acid (TFA) in dichloromethane (1 mL) for 12 h at room temperature. The product (H₂N-Gly-CBT) was purified by precipitation with cold diethyl ether. 1H NMR (400 MHz, CD₃OD) δ=3.86 (s, 2H), 7.62 (d, J=8.8 Hz, 1H), 8.08 (d, J=8.8 Hz, 1H), 8.62 (s, 1H).

H₂N-Gly-CBT (20 mg, 0.15 mmol) was added to a mixture of NHS-Dabcyl (37 mg, 0.1 mmol) and N,N-diisopropylethylamine (DIPEA, 33 μL, 0.2 mmol) in DMF (500 μL). The solution was stirred overnight at room temperature and the crude product was extracted with ethyl acetate. The combined organic phase was dried with Na₂SO₄ and evaporated. The crude product was redissolved in hexane and purified by silica gel column chromatography using 55% ethyl acetate in hexane as the eluant. Evaporation of the solvent produced Dabcyl-CBT as an orange solid (18 mg, 43% yield). JH NMR (400 MHz, CD₃OD) δ=3.14 (m, 1H), 7.71 (d, J=8.8 Hz, 1H), 8.19 (d, J=8.8 Hz, 1H), 8.72 (s, 1H). MALDI FT-ICR MS (m/z): calculated for C₂₅H₂₂N₇O₂S [M+H⁺] 484.1550; observed 484.1547.

### Synthesis of FAM-CO-SR⁶

5(6)-Carboxyfluorescein succinimidyl ester (48 mg) was mixed with sodium 2-mercaptoethanesulfonate (187 mg) in 4 mL of 1:1 (v/v) DMF/100 mM sodium borate (pH 8.7). The solution was stirred at room temperature for 2 h. The product was purified by reversed-phase HPLC and lyophilized to give a pure product (UPLC-MS: calculated for C₂₃H₁₇O₇S₂ [M+H⁺] 501.03; observed 501.07).

### Synthesis of Peptide Library

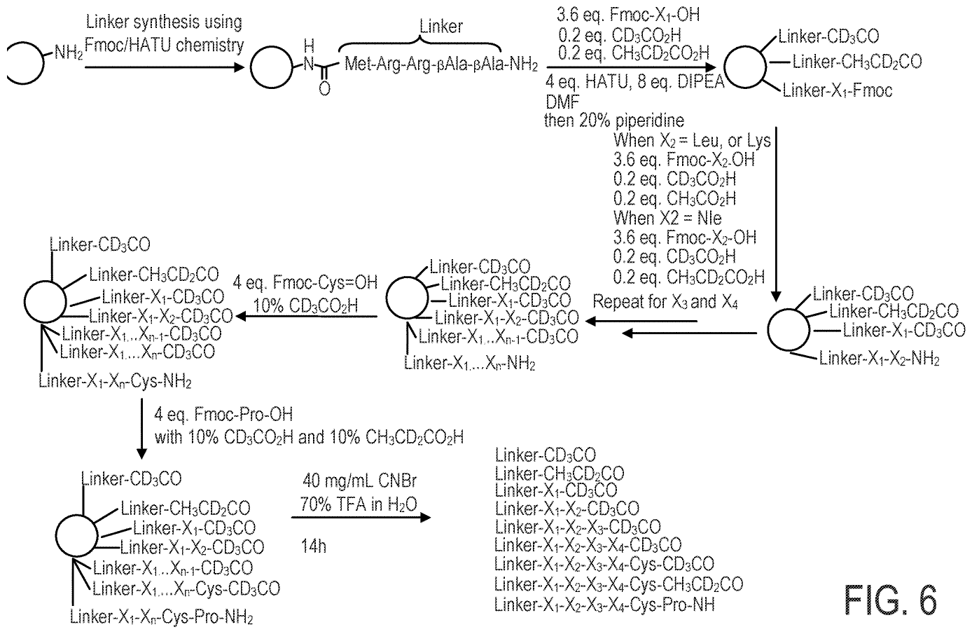

The peptide library was synthesized on PEGA resin (0.40 mmol/g loading) using standard Fmoc/HATU (FIG. 6). First, the common linker sequence, BAla-BAla-Arg-Arg-Met (SEQ ID NO: 85), was synthesized by using 4 eq. of Fmoc-amino acid, 4 eq. of HATU, 4 eq. of HOBt, and 8 eq. of DIPEA (coupling time=1 h). After removal of the Fmoc group from the second BAla with 20% piperidine for 5 min (twice), the library resin was suspended in DMF (20 mL/g) and split into 20 equal aliquots (by volume) and placed into 20 different reaction vessels (micro-spin columns). To each vessel, a different Fmoc-amino acid, other coupling reagents, and 5% (mol/mol; relative to Fmoc-AA) each for CD₃CO₂D and CH₃CD₂CO₂H (capping agents) were added, and the coupling reaction was allowed to proceed for 1 h. After exhaustive washing, the resin from the 20 reaction vessels was pooled together and the Fmoc group was deprotected with 20% piperidine. The resin was again split into 20 equal aliquots and subjected to the next round of peptide synthesis reaction. To differentiate amino acids of the same molecular weight during peptide sequence determination by mass spectrometry,²⁴ 5% (mol/mol) CH₃CO₂H was also added to the coupling reactions of Leu and Lys, while 5% CH₃CD₂CO₂H was also added to the coupling reaction of Ile. After the four random positions were synthesized, the resin was pooled, treated with 20% piperidine (to remove the N-terminal Fmoc), and cysteine and proline residues were coupled by using the standard coupling condition described above. After removal of the N-terminal Fmoc group, the library peptides were deprotected by treating the resin with modified Reagent K (2.5% triisopropylsilane, 2.5% H₂O, 2.5% 2,2′-(ethylenedioxy) diethanethiol, 2.5% phenol, in TFA) for 3 h. The library was suspended in DMF and stored at −20° C. until use.

### Library Screening

A portion of the peptide library (20 mg) was transferred into a disposable Bio-Spin column (2.0 mL). The resin was washed with DMF and a ProAP screening buffer [PBS (pH 7.4) containing 1 mM DTT] three times each. The resin was suspended in the screening buffer and treated with 0-1.0 μM ProAP (total reaction volume=1 mL) at room temperature for 30 min. The reaction was terminated by removing the enzyme solution (via filtration), and the resin was washed with 9:1 (v/v) DMSO/PBS (pH 7.4) containing 1 mM TCEP. The resin was treated for 2 h with 5.0 equiv. of Dabcyl-CBT in 9:1 (v/v) DMSO/PBS (pH 7.4) containing 1 mM TCEP. The resin was washed with ddH₂O (three times) and transferred into a Petri dish with H₂O. The solution was acidified to pH~1 with HCl, and 30 most intensely red-colored beads were manually (and immediately) removed from the library with a micropipette under a dissecting microscope. Thirty colorless beads were randomly selected from the 1-μM reaction. The beads were placed into individual microcentrifuge tubes, and each treated overnight with 50 μL of 100 mg/mL CNBr in 70% TFA at room temperature. The next day, the solution was evaporated to dryness in vacuo. The released peptide in each tube was dissolved in 5 μL of 0.1% TFA in H2O. For MS analysis, a 1-μL aliquot of the TFA solution was mixed with 1 μL of a saturated solution of α-cyano-4-hydroxycinnamic acid, and 1 μL of the resulting mixture was applied to the spectrometer plate. Mass spectrometry was performed on a Bruker ultrafleXtreme MALDI-TOF-TOF spectrometer. The data obtained were analyzed by Data Analysis software (Bruker).

### Peptide Synthesis

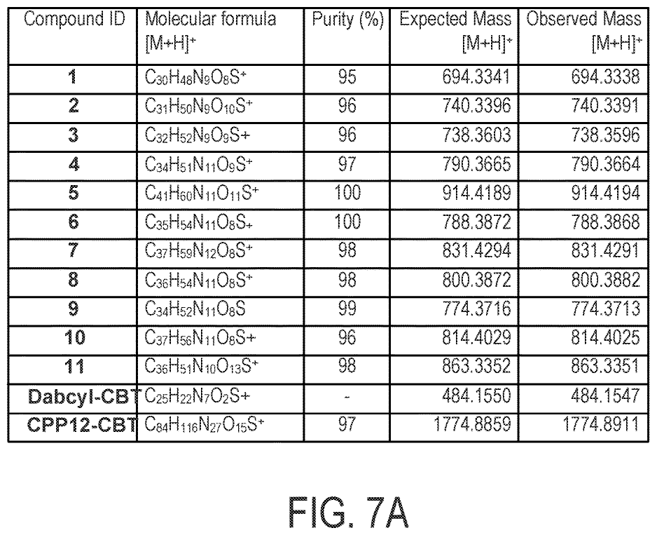

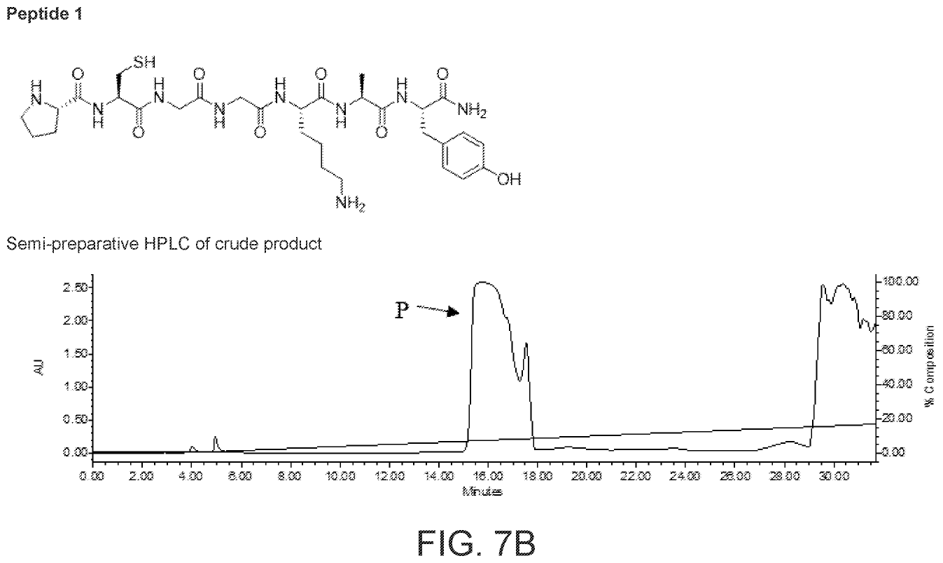

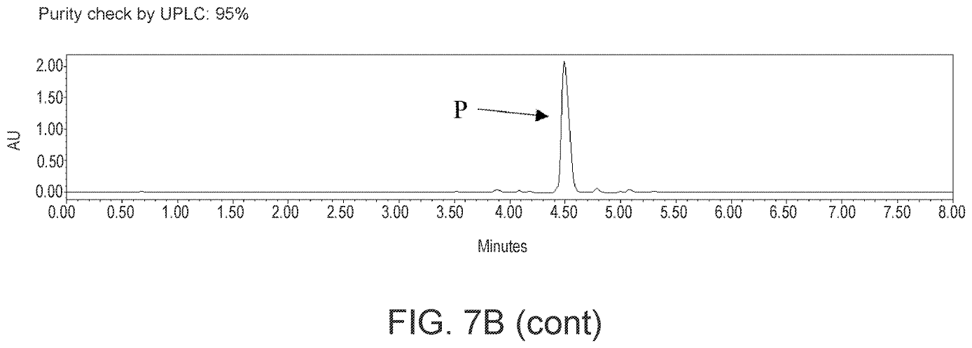

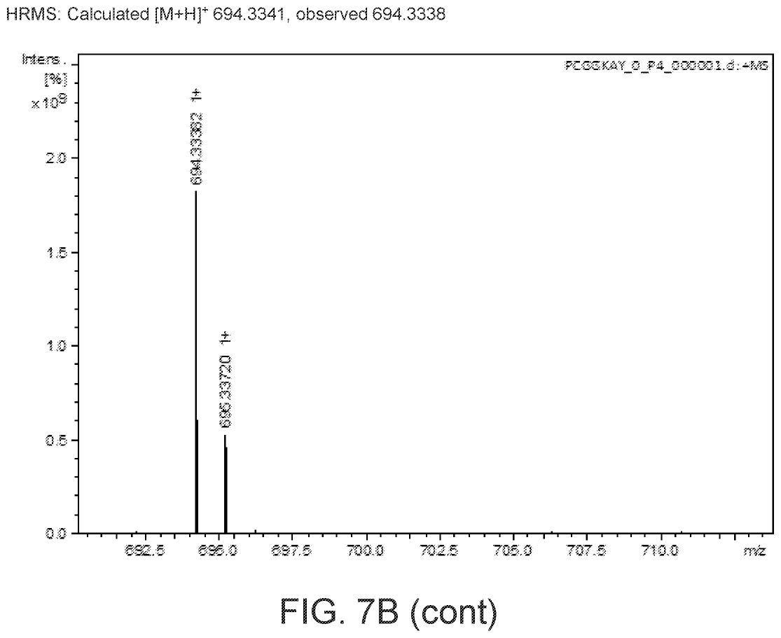

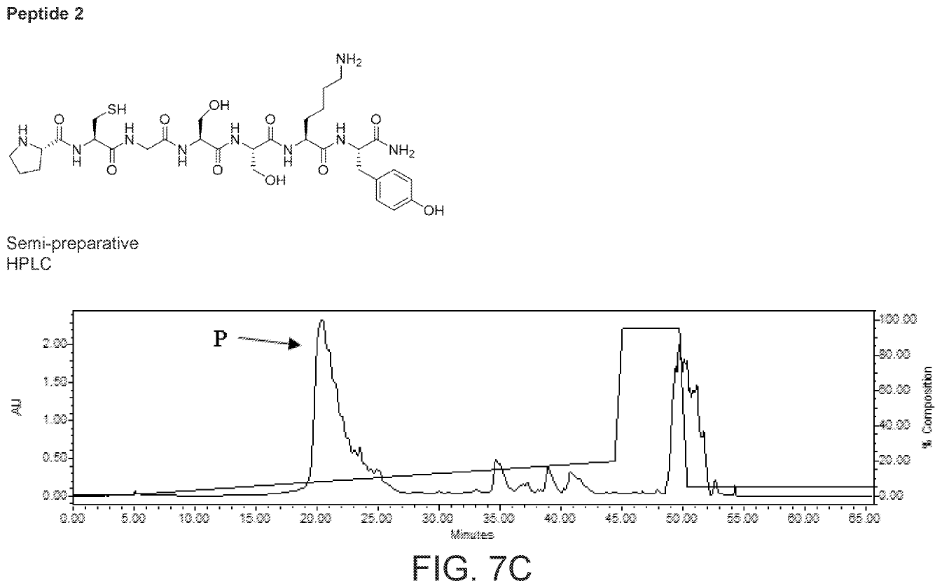

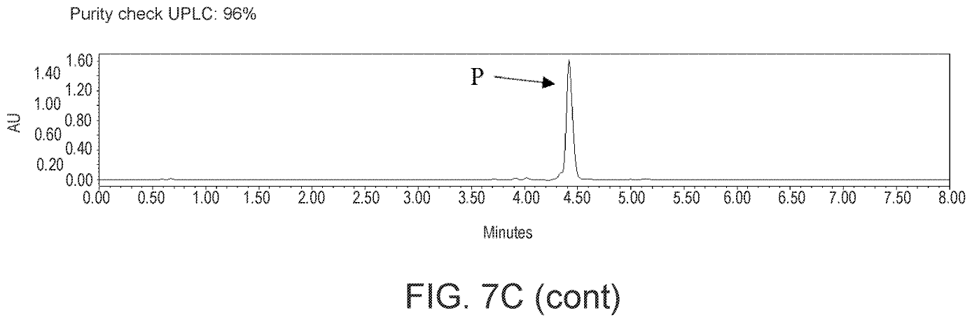

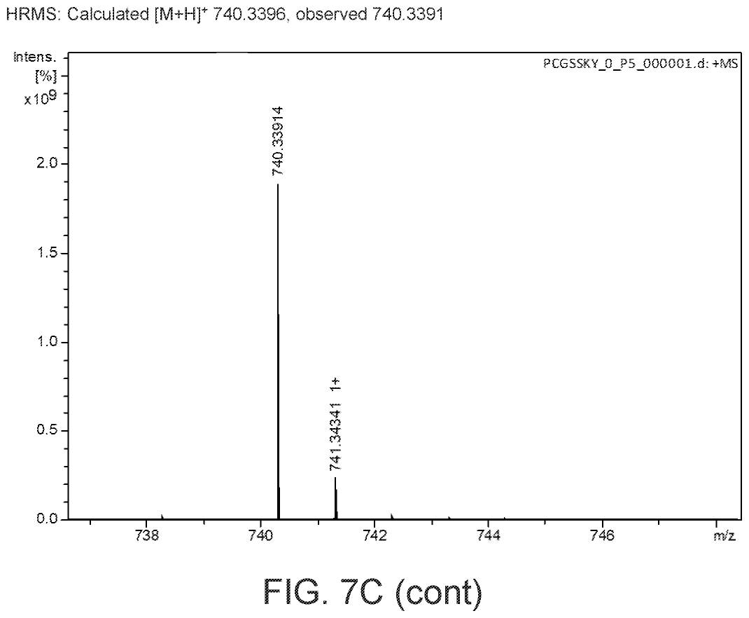

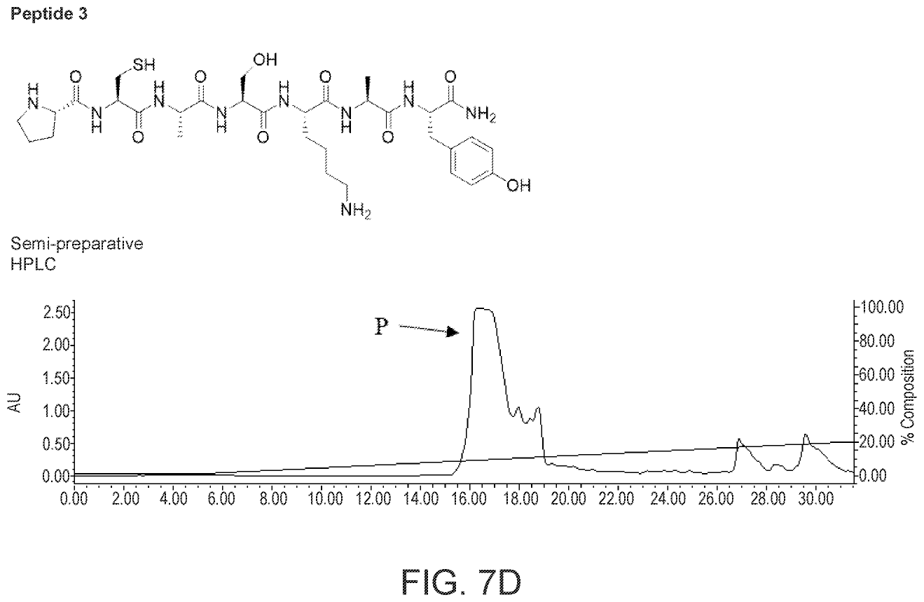

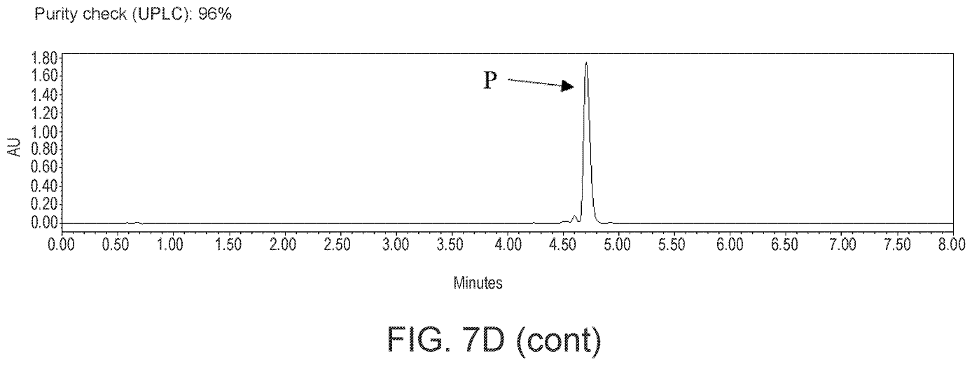

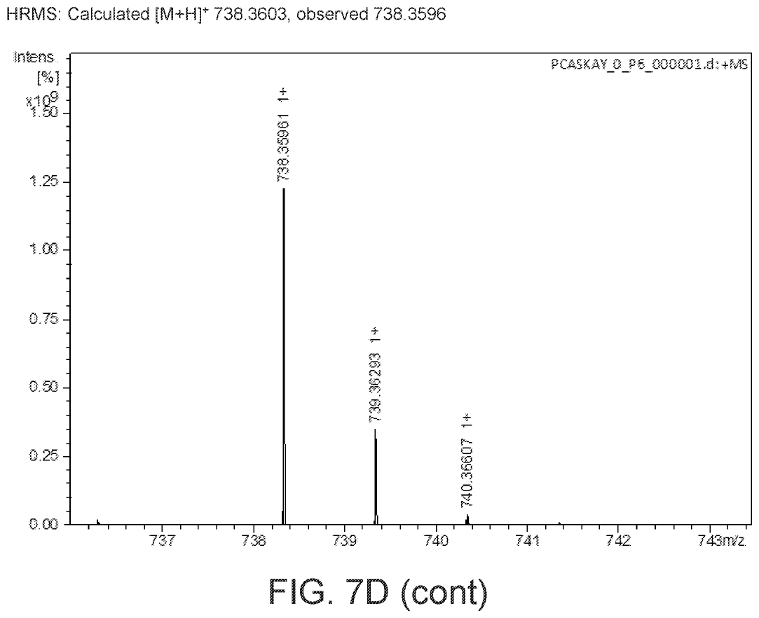

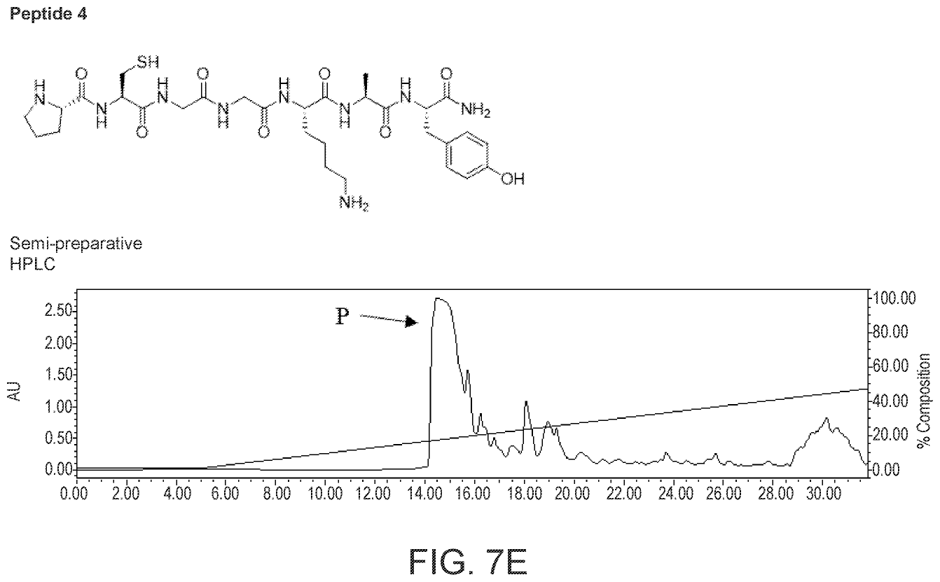

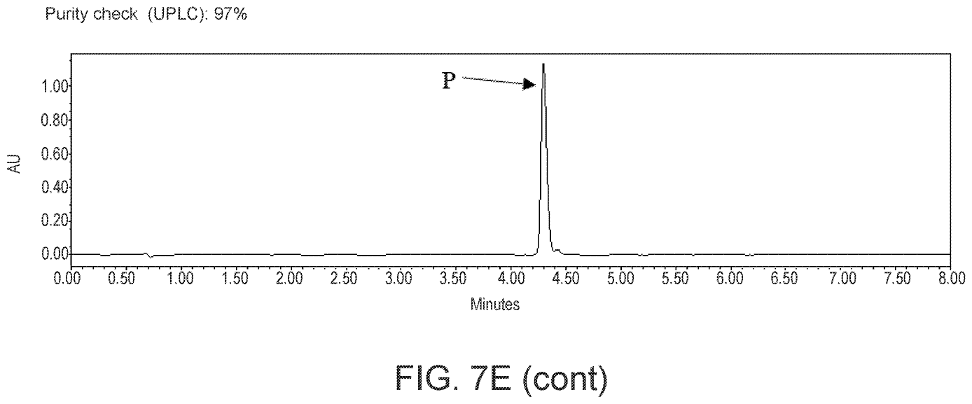

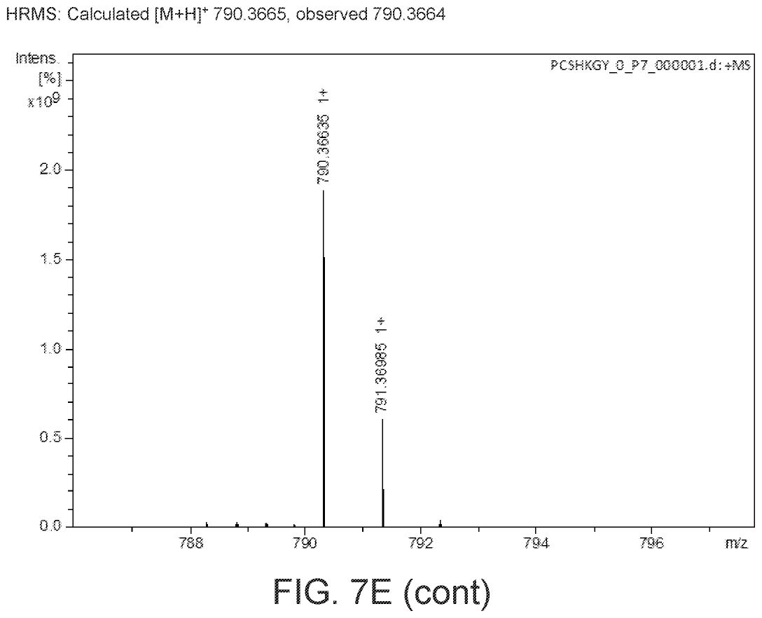

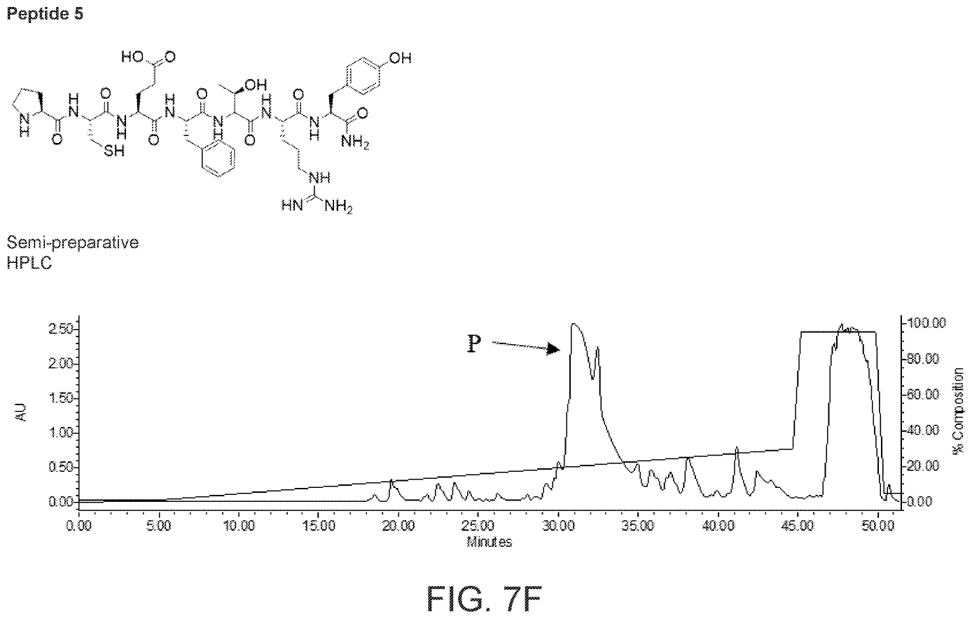

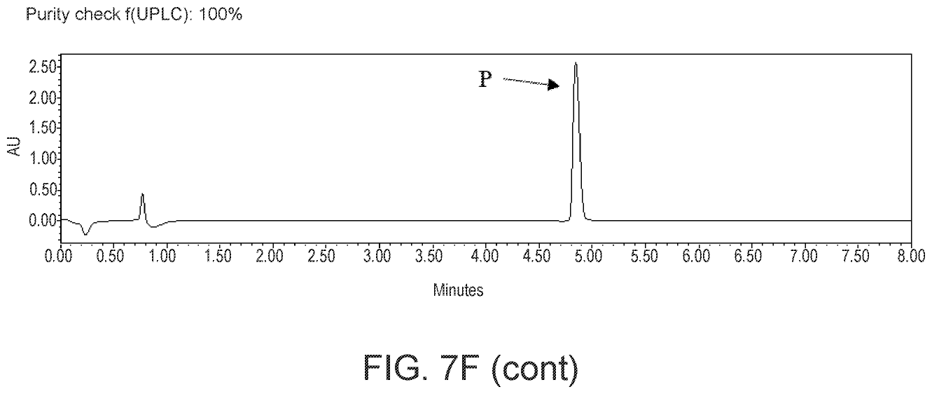

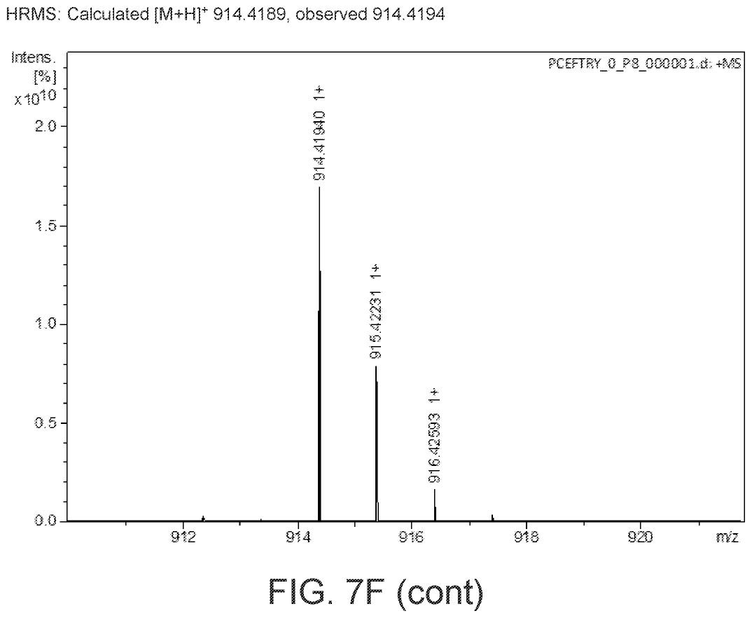

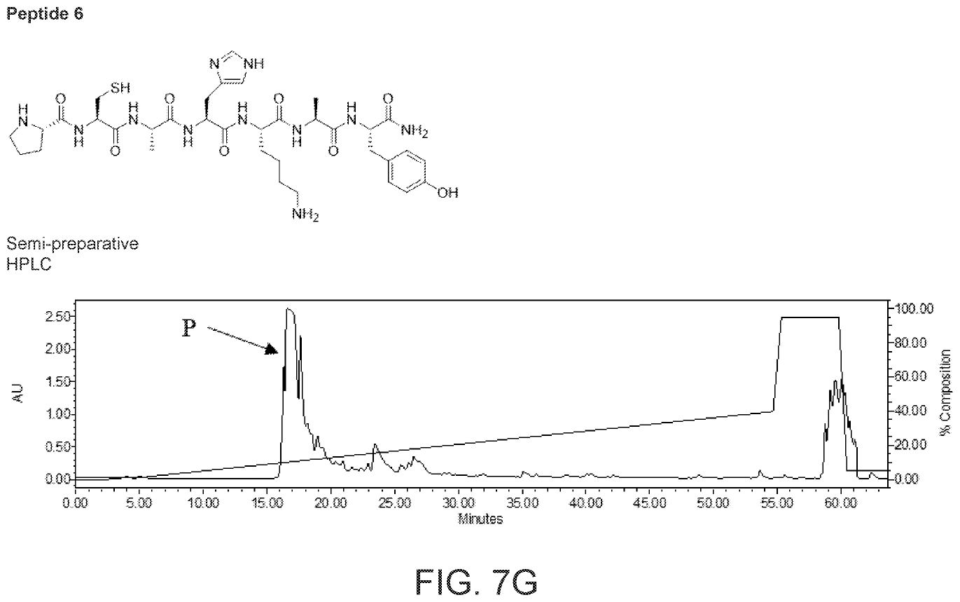

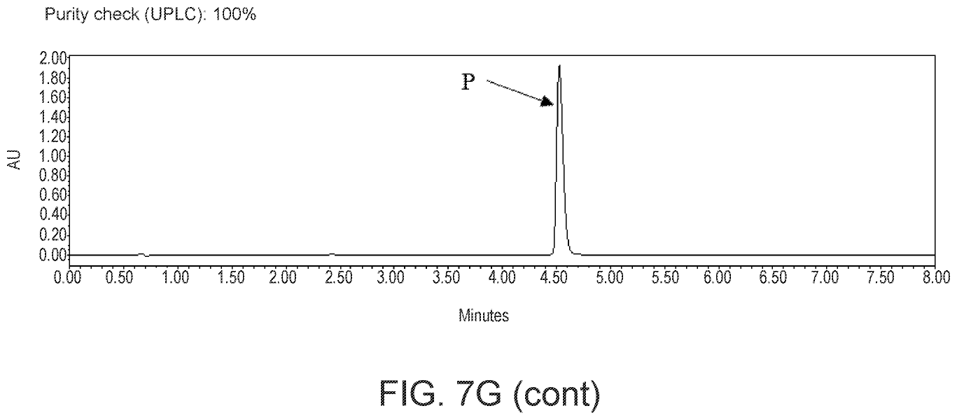

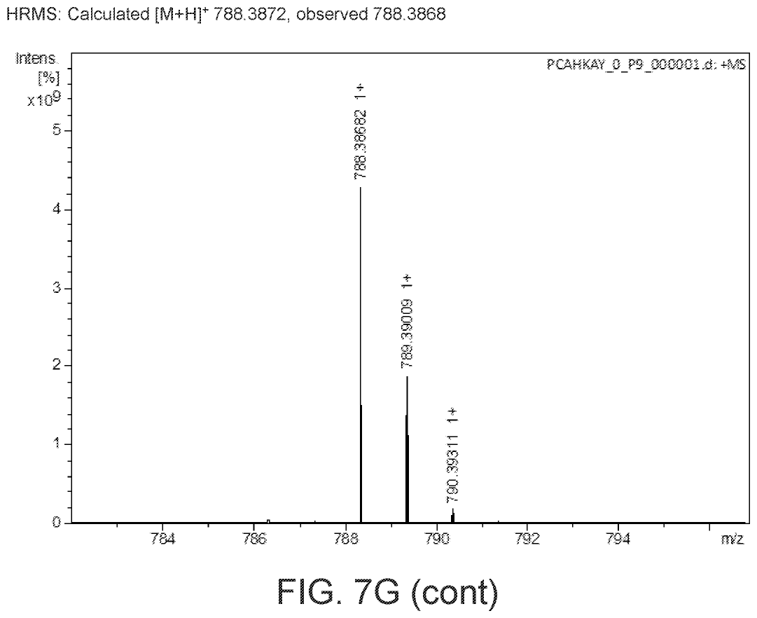

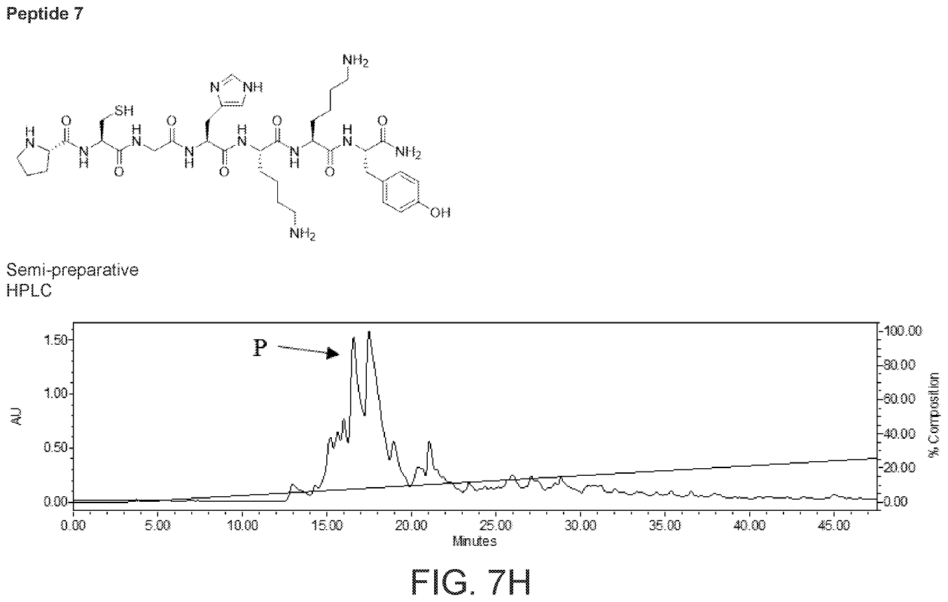

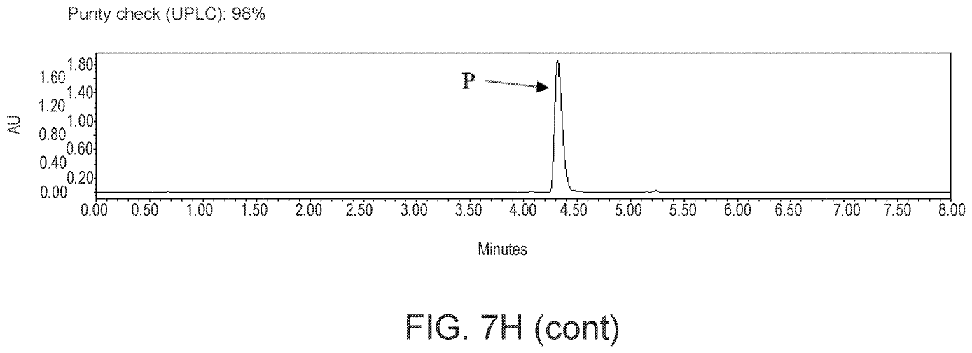

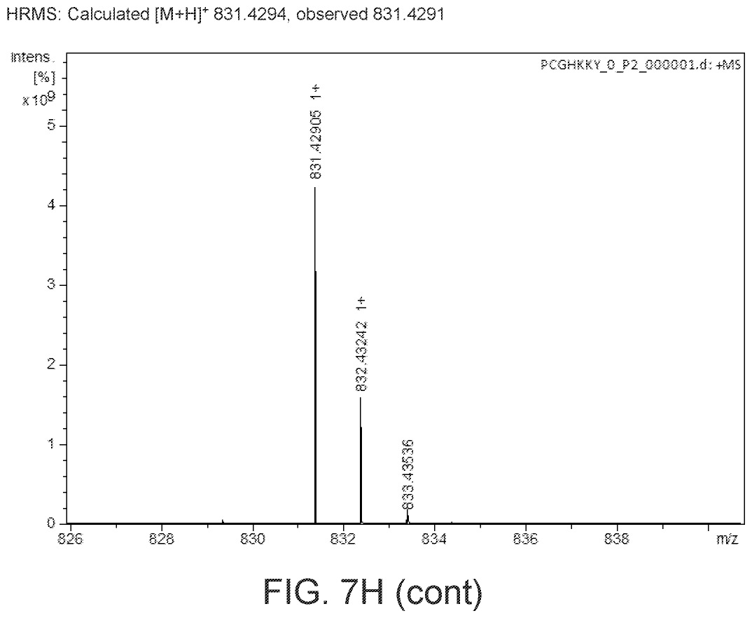

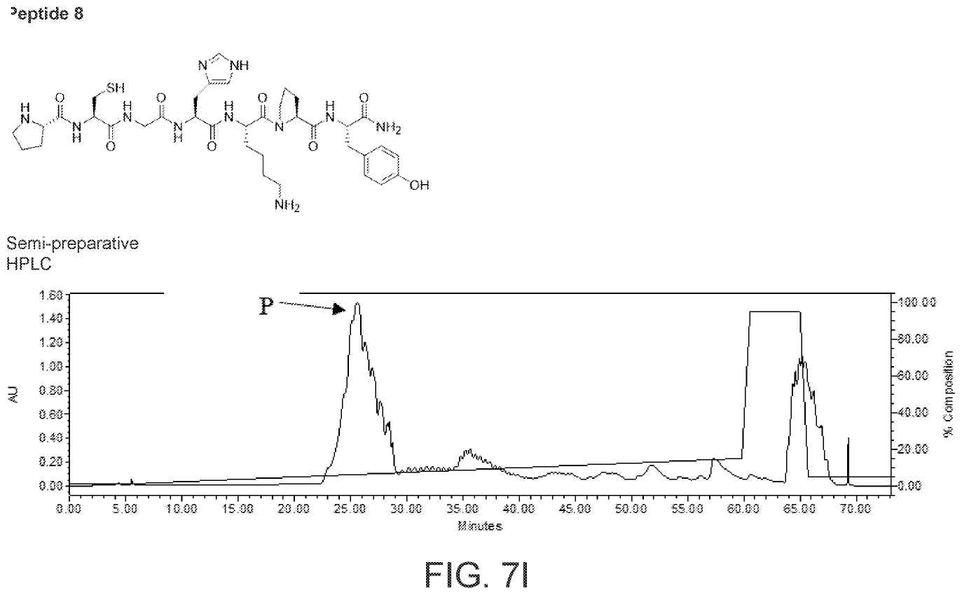

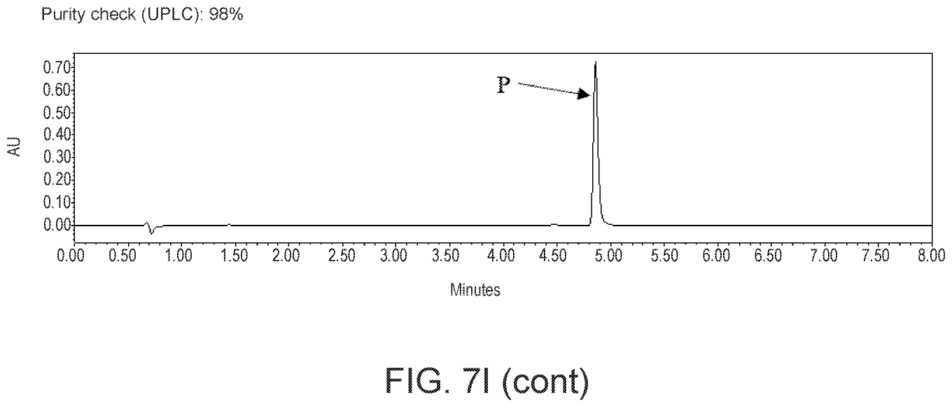

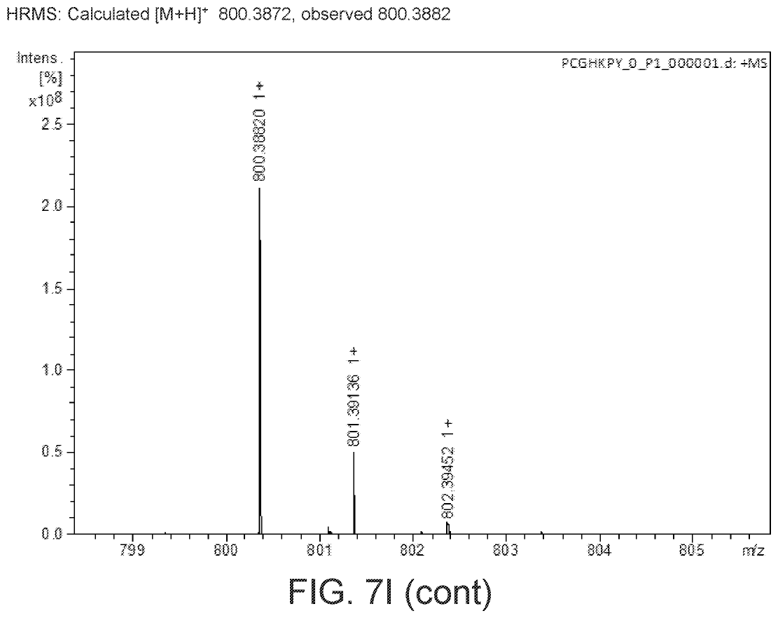

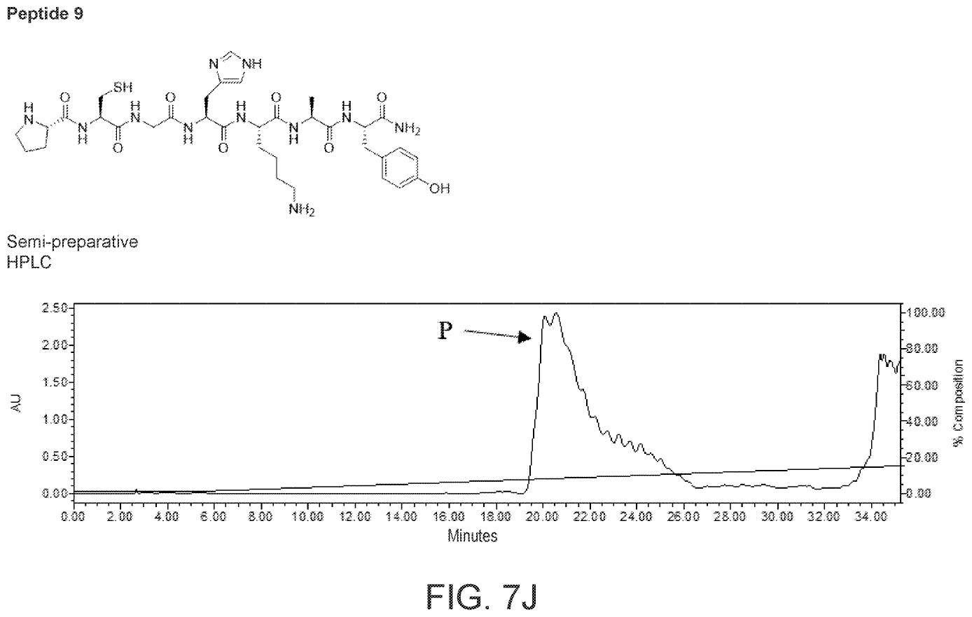

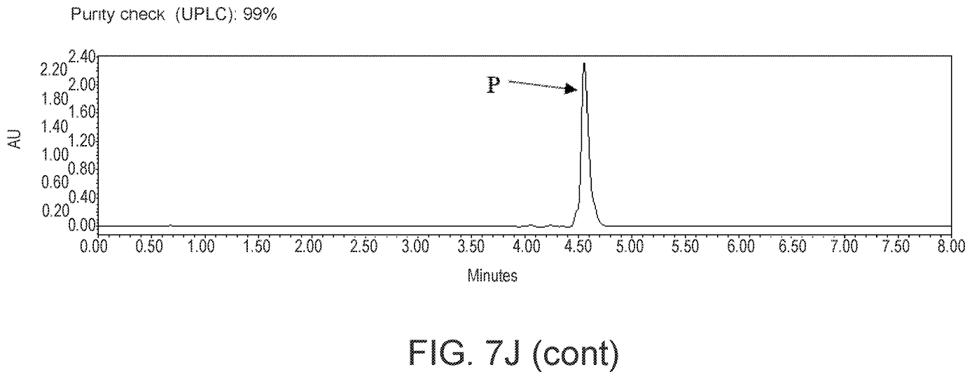

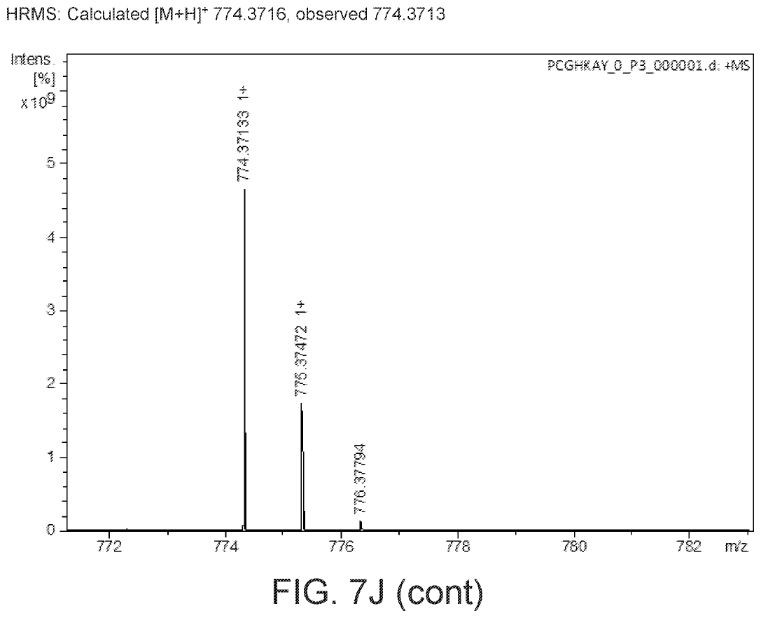

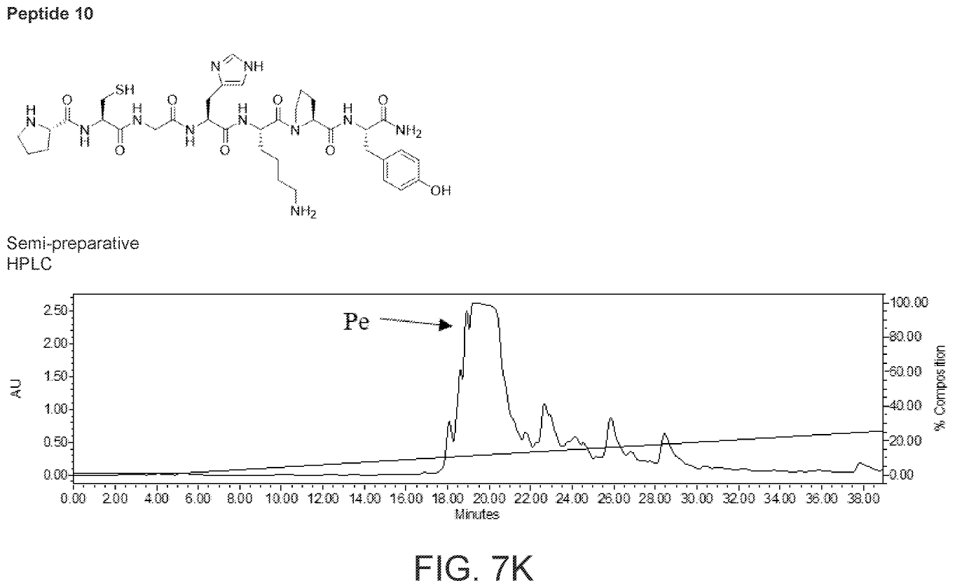

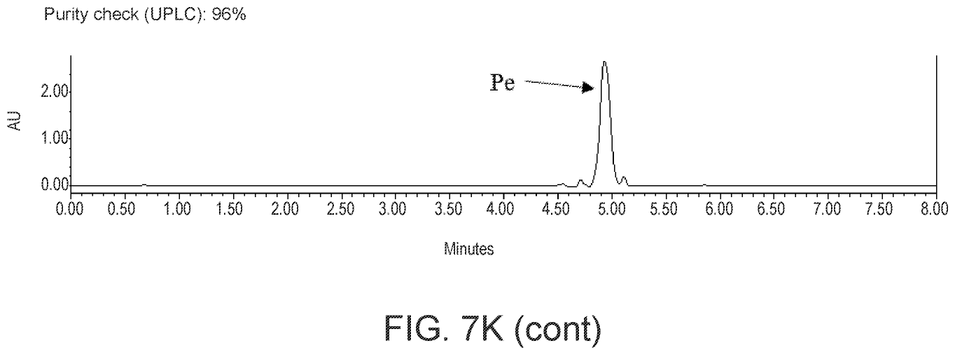

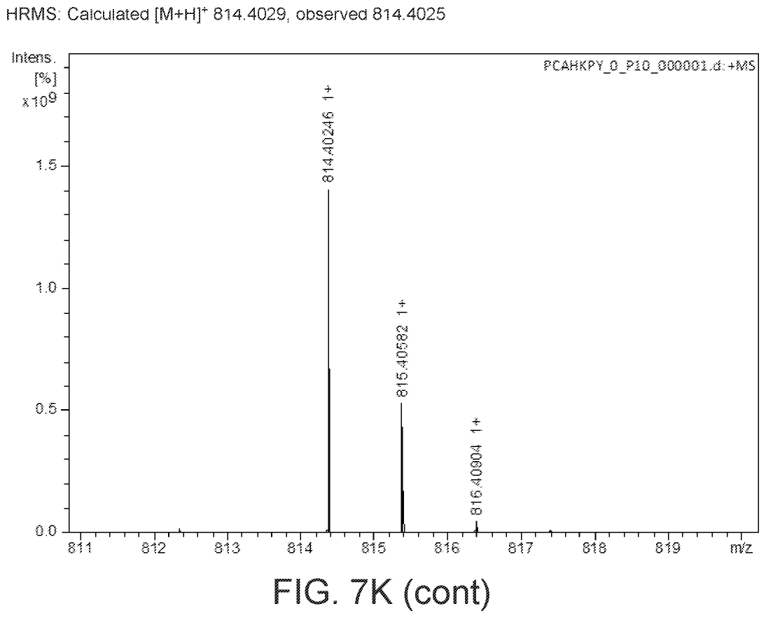

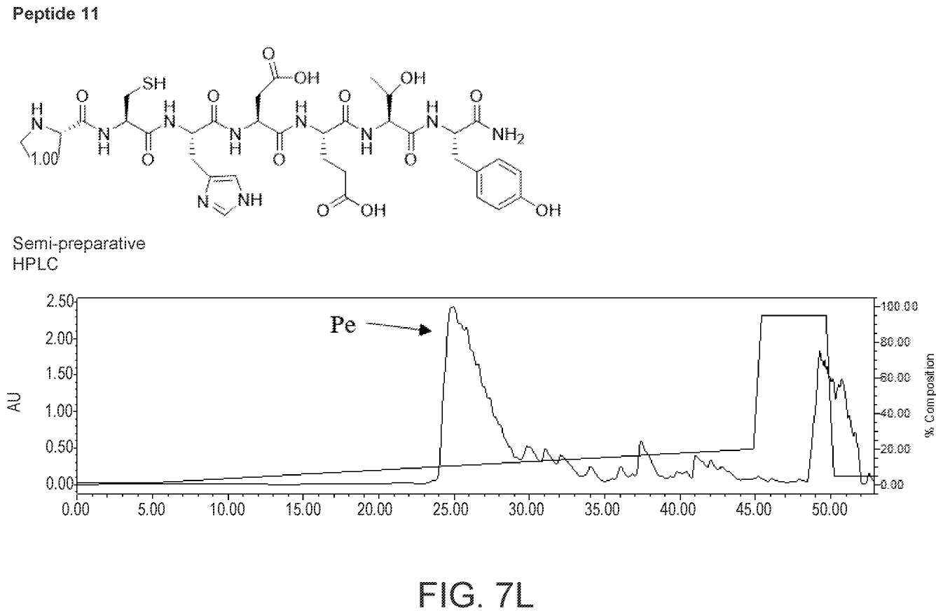

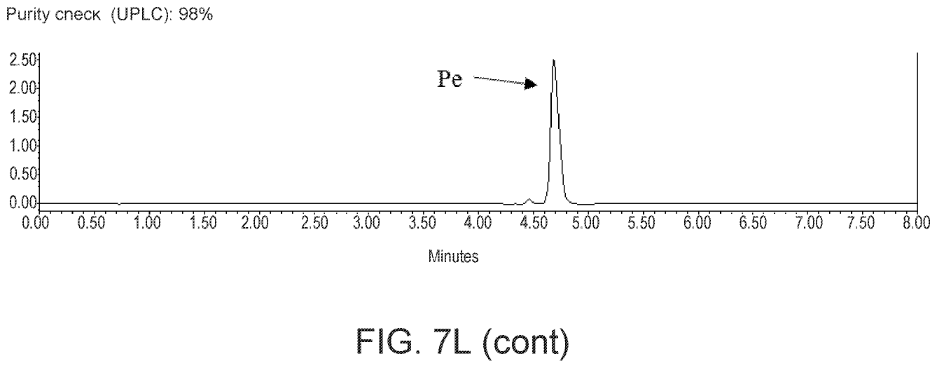

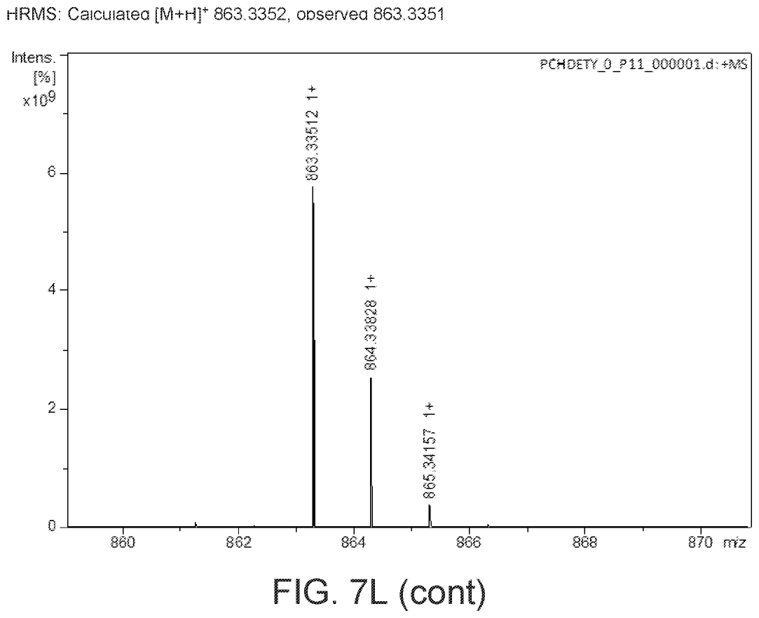

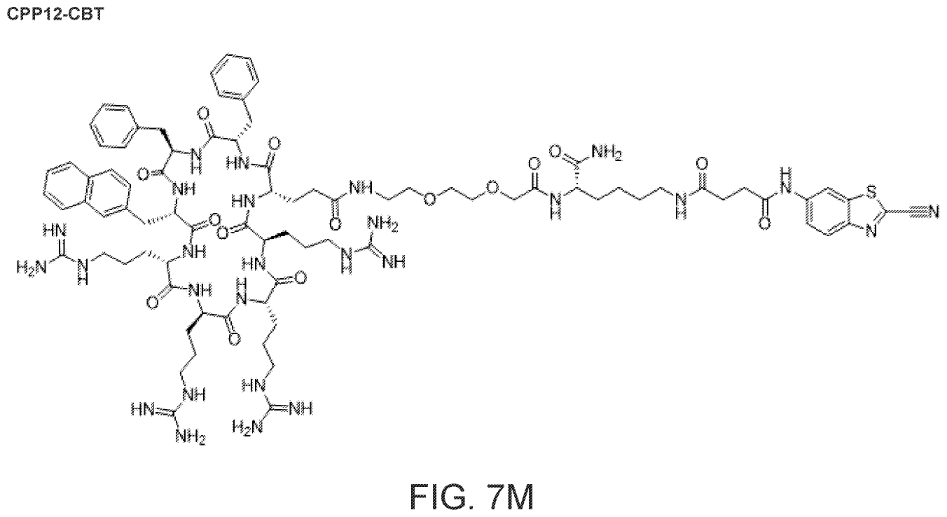

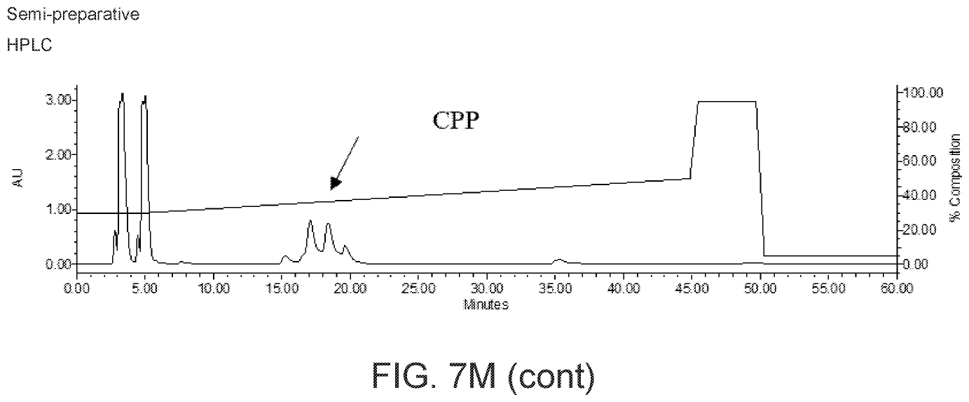

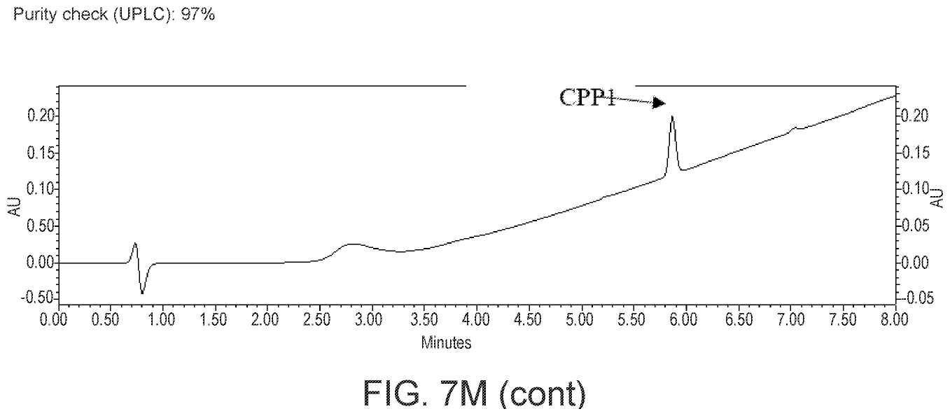

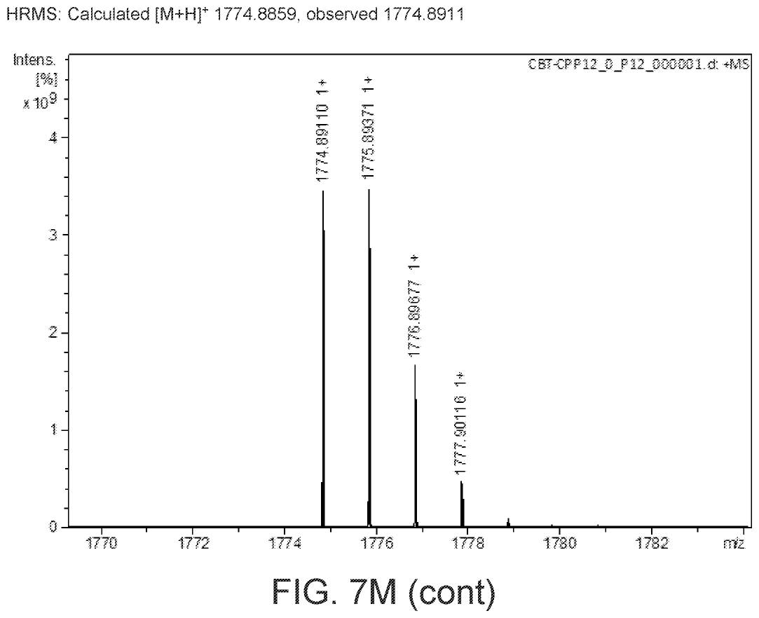

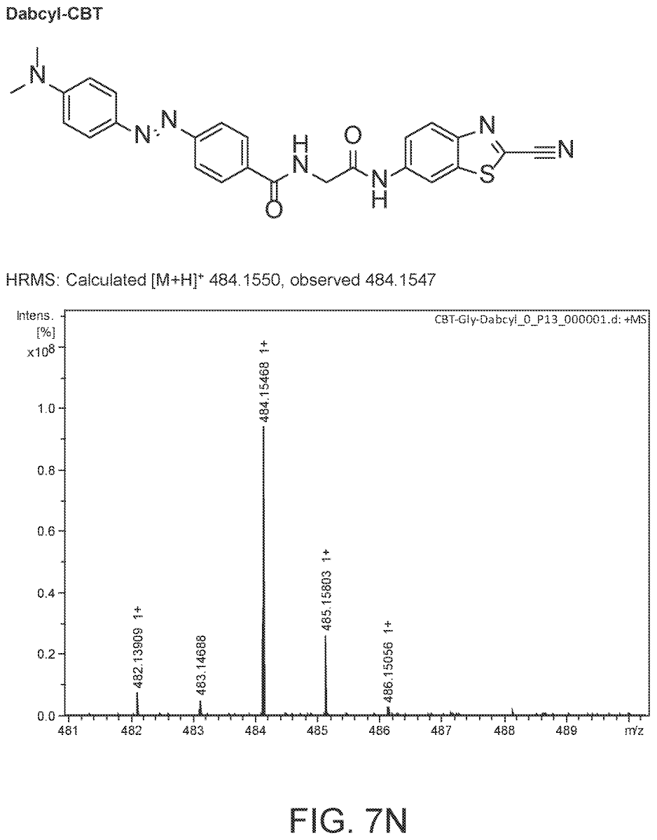

Peptides were synthesized on a CEM Liberty Blue automated microwave peptide synthesizer using Fmoc/DIC chemistry. Peptide synthesis was carried out on Rink amide resin (50-100 mesh, 0.54 mmol/g, Chem-Impex) at a 0.05 mmol scale. Each coupling reaction was performed by using 0.2 M Fmoc-amino acid in DMF and DIC/Oxyma Pure (0.5 M in DMF) at 90° C. (20 W) for 4 min (twice). The N-terminal Fmoc-group was removed using 20% piperidine (v/v) in DMF. Peptides were cleaved from the resin and deprotected by treatment with 90:2.5:2.5:2.5:2.5 (v/v) TFA/H₂O/1,4-dimethoxybenzene/TIPS/2,2′-(ethylenedioxy) diethanethiol for 3 h. The crude peptide was triturated three times with cold ethyl ether and purified to ≥95% purity by reversed-phase HPLC equipped with a Waters C18 column, which was eluted with a linear gradient of acetonitrile (0-30%) in ddHO (containing 0.05% TFA). The authenticity of the peptides was confirmed by MALDI FT-ICR MS (FIGS. 7A-7M).

### Synthesis of CPP12-CBT

CPP12-miniPEG-Lys was synthesized and purified by reversed-phase HPLC as previously described.²⁵ 2-Cyano-6-aminobenzothiazole (42 mg, 0.24 mmol) and succinic anhydride (235 mg, 2.4 mmol) were dissolved in 400 μL of THE and 100 μL DMF and allowed to react overnight at 60° C. The product was purified by reversed-phase HPLC. Next, CPP12-miniPEG-Lys (3 mg) was dissolved in 10 μL of DMF, to which a catalytic amount of 4-dimethylaminopyridine (~0.1 mg), DIC (3 μL; 2 mg), and succinyl-CBT (3 mg in 10 μL of DMF) were added. This reaction was allowed to proceed for 1 h and the crude product was purified by reversed-phase HPLC. MALDI FT-ICR MS (m/z): calculated for C₈₄H₁₁₅N₂₇O₁₅S [M+H⁺] 1774.8859; observed 1774.8911.

### Molecular Cloning

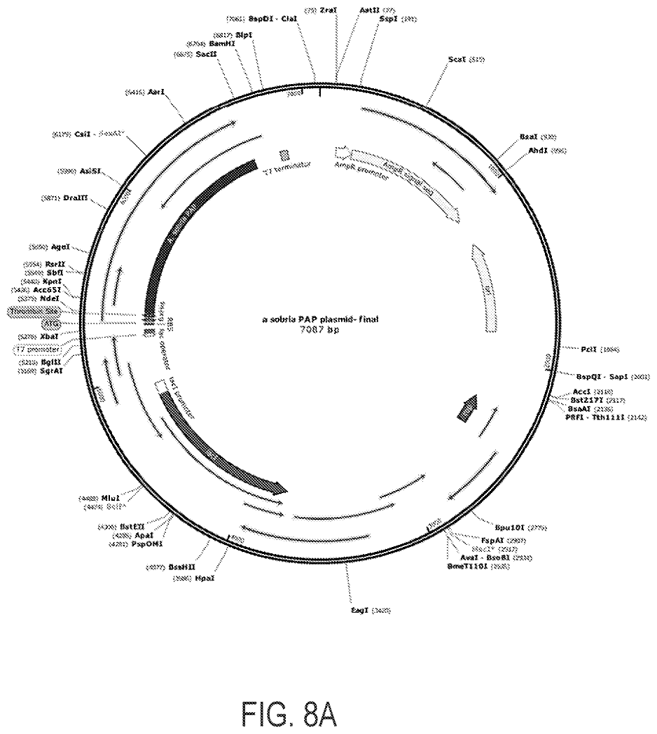

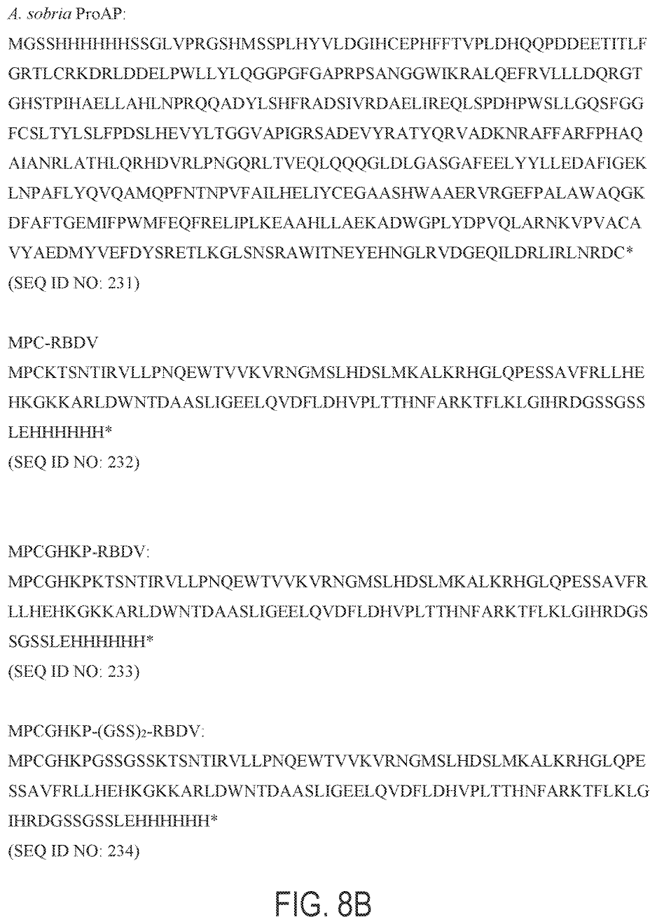

The coding sequence of A. sobria ProAP¹⁹ plus an N-terminal six-histidine tag was chemically synthesized and cloned into prokaryotic expression vector pET-15b(+) to generate plasmid pET-15b(+)-Hiss-ProAP (FIGS. 8A-8B) (SEQ ID NO: 86) by Genscript Biotech (Piscataway, NJ). Similarly, the coding sequence of RBDV (SEQ ID NO: 87) bearing an N-terminal Met-Pro-Cys motif and a C-terminal six-histidine tag was chemically synthesized and cloned into prokaryotic expression vector pET-22b(+) to give plasmid pET-22b(+)-RBDV-His₆ (FIGS. 8A-8B) (SEQ ID NO: 88). To generate an expression plasmid for RBDV (SEQ ID NO: 87) that contains an N-terminal MPCGHKP (SEQ ID NO: 89) sequence [pET-22b(+)-MPCGHKP-RBDV-His₆] (SEQ ID NO: 90), plasmid pET-22b(+)-RBDV-Hiss DNA (SEQ ID NO: 88) was amplified by one-step polymerase chain reaction (PCR) using DNA primers 5′-GGTCACAAACCGAAGACCAGCAATACCATCCG-3′ (SEQ ID NO: 91) and S′-CGGTTTGTGACCGCACGGCATATGTATATCTCCT-TCTTAAA-3′ (SEQ ID NO: 92) and following published protocols.²⁶ Similarly, to generate an expression plasmid for RBDV (SEQ ID NO: 87) that contains an N-terminal MPCGHKPGSSGSS sequence [pET-22b(+)-MPCGHKP-GSSGSS-RBDV-His₆], plasmid pET-22b(+)-MPCGHKP-RBDV-His₆ (SEQ ID NO: 88) DNA was amplified by one-step PCR using oligonucleotides 5′-GGTTCTTCTGGTTCTTCTAAGACCAGCAATACCATCCGTG-3′ (SEQ ID NO: 93) and 5′-AGAAGAACCAGAAGAACCCGGTTTGTGACCGCACG-3′ (SEQ ID NO: 94) as primers. The DNA products were treated with restriction endonuclease DpnI (to digest the methylated plasmid template) and used to transform Escherichia coli DH5a cells, from which the desired DNA plasmids were generated through homologous recombination, amplified, and purified. The authenticity of the plasmids was confirmed by Sanger sequencing the entire coding regions of proteins of interest.

### Protein Expression and Purification

E. coli BL21(DE3) cells transformed with plasmid pET-15b(+)-His₆-ProAP were grown in Luria Broth (LB) media supplemented with 75 μg/mL ampicillin at 37° C. until OD₆₀₀ reached 0.6-0.8. Protein expression was induced with 500 μM IPTG at 30° C. for 6 h. The cells were pelleted by centrifugation at 5,000 rpm (GS-3 rotor), 4° C. for 20 min, and stored at −80° C. Cell lysis was performed by suspending the cell pellets in lysis buffer (50 mM Tris, pH 7.5, 300 mM NaCl, 2 mM β-mercaptoethanol, 0.2 mg/mL lysozyme, 3 cOmplete™ protease inhibitor tablets (Roche)) and stirring at 4° C. for 30 min. The lysate was sonicated at 70% amplitude on ice for 1 min (in short pulses of 2 sec with pauses of 8 sec). The crude lysate was centrifuged at 14,000 rpm (SS-34 rotor) and 4° C. and the supernatant was loaded onto a HisPur Cobalt column over 1 h. The column was sequentially washed with 50 mM Tris, pH 7.5, 300 mM NaCl, 3 mM β-mercaptoethanol and then the same buffer containing 20 mM and 30 mM imidazole. The bound protein was eluted with 20 mM Tris (pH 7.5), 150 mM NaCl, and 150 mM imidazole. Fractions containing pure ProAP were concentrated by using Amicon Ultra-15 centrifugal filter units (MWCO: 30 kDa). Protein concentration was determined by the Bradford assay (Bio-Rad) and the typical yield was 1.6 mg of enzyme per liter of culture. After the addition of 20% (v/v) glycerol (final concentration), the protein was aliquoted, quickly frozen, and stored at −80° C.

Expression and purification of RBDV proteins (SEQ ID NO: 87) were similarly carried out but with the following modifications. Induction of protein expression involved 100 μM IPTG for 18 h at 18° C. Cell lysis was performed in 50 mM Tris, pH 7.5, 150 mM NaCl, 3 mM β-mercaptoethanol, 0.2 mg/mL lysozyme, 3 mM PMSF, and 2 cOmplete™ protease inhibitor tablets (Roche)) for 15 min at 4° C. The crude lysate was fractionated on an ÄKTA explorer FPLC system (Amersham Pharmacia Biotech) equipped with a “HisTrap FF 5 mL” nickel affinity column (GE Healthcare). The column was washed with 50 mM Tris, pH 7.5, 150 mM NaCl, 3 mM β-mercaptoethanol, and 30 mM imidazole and eluted with a linear gradient of 30-300 mM imidazole in 50 mM Tris, pH 7.5, 150 mM NaCl. The protein was concentrated to ~5 mg/mL and buffer exchanged into 50 mM Tris, pH 7.5, 150 mM NaCl to remove the imidazole in an Amicon Ultra-15 centrifugal filter units (MWCO: 10 kDa).

### ProAP Activity Assay

A typical assay reaction (total volume of 100 μL) contained PBS (10 mM Na₂HPO₄, 1.8 mM KH₂PO₄, 137 mM NaCl, 2.7 mM KCl, 2 mM DTT pH 7.4) and 0-500 μM peptide substrate. The reaction was initiated by the addition of ProAP (final concentration of 0.5-25 nM) and quenched after 5-30 min by the addition of 100 μL of 10% TFA. The reaction mixture was centrifuged at 15,000 rpm in a microcentrifuge for 5 min, and the clear supernatant was analyzed on a Waters Acquity UPLC-MS equipped with a BEH C18 column (1.7 μm, 2.1 mm I.D., 100 mm length). The column was eluted with a linear gradient of acetonitrile (0-25% over 8 min) in water containing 0.05% TFA. The percentage of substrate-to-product conversion was determined by comparing the peak areas of the remaining substrate and the reaction product (monitored at 280 nm) and kept at ≤20%. The initial rates were calculated from the conversion percentages and plotted versus [S]. Data fitting against the Michaelis-Menten equation V=Vₘₐₓ[S]/(K_{M}+[S]) or the simplified equation V=k_{cat}[E][S]/K_{M} (when K_{M}>>[S]) gave the kinetic constants k_{cat}, K_{M}, and/or k_{cat}/K_{M}.

ProAP Activity Assay against Protein Substrates

ProAP, PCGHKP-RBDV (SEQ ID NO: 95), and PCGHKP-(GSS)₂-RBDV (SEQ ID NO: 96) were exhaustively dialyzed against 20 mM Tris, pH 8.5, 100 mM NaCl, 10 mM DTT, and 1 mM EDTA (twice 4 L for 4 h and 4 L overnight) to remove any divalent metal ions (e.g., Co²⁺ and Ni²⁺). PCGHKP-(GSS)₂-RBDV (25 μM) (SEQ ID NO: 96) was incubated with ProAP (1 μM) at 37° C. in 20 mM Tris, pH 8.5, 100 mM NaCl, 10 mM DTT, and 1 mM EDTA. At various time points (0, 1, 5, 15, 60, and 180 min), aliquots (40 μL) of the reaction were withdrawn and quenched by the addition of 40 μL of 10% TFA. The resulting samples were analyzed by SDS-PAGE and MALDI-FT-ICR mass spectrometry. Treatment of PCGHKP-RBDV (SEQ ID NO: 95) with ProAP was similarly carried out, except that the aliquots were withdrawn at different time points (0, 15, 60, 180, 360, and 480 min).

N-Terminal Labeling with Fluorescein

Peptide (100 μM) was incubated with ProAP (4 μM) in 20 mM Tris, pH 8.5, 100 mM NaCl for 1 h at 37° C. One half of the reaction (40 μL) was set aside for later UPLC-MS analysis, while the other half was mixed with 40 μL of FAM-CO-SR (400 μM), sodium 2-mercaptoethanesulfonate (2 mM), and TCEP (2.5 mM) in PBS (H 7.5). The mixture was incubated at room temperature for 5 h and analyzed by UPLC-MS.

Exhaustively dialyzed PCGHKP-(GSS)₂-RBDV (30 μM) (SEQ ID NO: 96) was incubated with ProAP (1.5 or 0 μM) in 20 mM Tris, pH 8.5, 100 mM NaCl, 10 mM DTT, and 1 mM EDTA for 1 h at 37° C. The reaction mixture was dialyzed against 1 L of PBS (pH 7.5) containing 1 mM TCEP and 1 mM EDTA. One half of the reaction was mixed with an equal volume of 50 mM sodium phosphate buffer (pH 7.5) containing 8 equivalents of FAM-CO-SR, 2.5 mM TCEP, 1 mM EDTA, and 40 equivalents of 2-mercaptoethanesulfonate (total volume 80 μL). The reaction was allowed to proceed at room temperature for 5 h and analyzed by SDS-PAGE and MALDI-FT-ICR mass spectrometry.

N-Terminal Conjugation of Proteins with CPP12

PCGHKP-(GSS)₂-RBDV (SEQ ID NO: 96) was dialyzed and treated with ProAP as described above. Half of the protein (40 μL) was set aside for later analysis while the other half was mixed with an equal volume of PBS (pH 7.4) containing 10% DMSO, 75 AM CPP12-CBT (5 equiv), 2 mM TCEP, and 1 mM EDTA. The reaction was allowed to proceed at room temperature for 2 h and analyzed by SDS-PAGE and MALDI-FT-ICR mass spectrometry.

### Results and Discussion

### Design Strategy

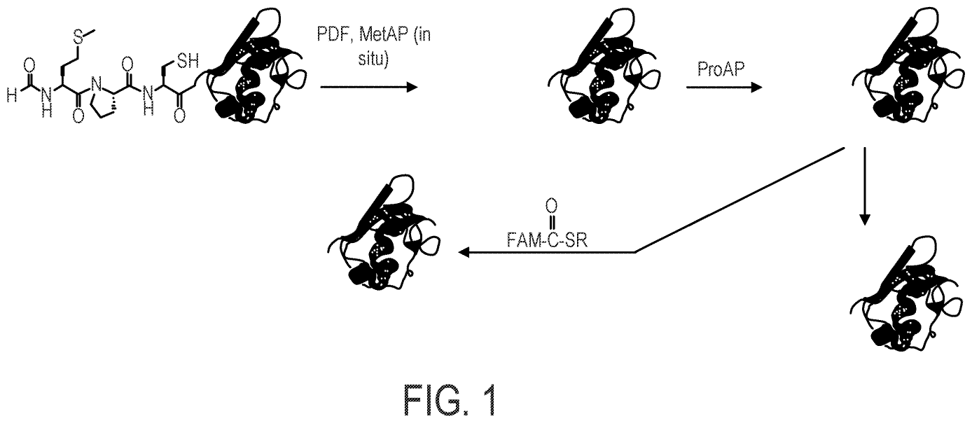

Our strategy involves the addition of a tripeptide sequence, Met-Pro-Cys, to the N-terminus of a protein of interest (FIG. 1). According to the substrate specificity of PDF¹⁵ and MetAPs,^{16,17} the N-terminal formyl-Met is efficiently removed in situ by the endogenous PDF and MetAP during expression in E. coli. Similarly, the N-terminal Met is completely removed by MetAPs when the protein is produced in eukaryotic cells.¹⁷ The N-terminal proline serves as a de facto protecting group, preventing the penultimate cysteine residue from modification by intracellular aldehydes and ketones,⁷ cleavage by most aminopeptidases, or N-terminal acetylation⁹ (when the protein is expressed in eukaryotic hosts). After purification, the N-terminal proline can be removed in vitro by treating the protein with a prolyl aminopeptidase (ProAP). The resulting N-terminal cysteine can then be specifically modified with an aldehyde/ketone,⁴ a thioester,⁵ or a CBT derivative.⁶

Identification of Optimal Substrates of Aeromonas sobria ProAP

Our initial attempt to remove the N-terminal proline from recombinant proteins containing an N-terminal Pro-Cys sequence by Bacillus coagulans ProAp¹⁸ was unsuccessful, because B. coagulans ProAP prefers short peptides (e.g., Pro-Xaa dipeptides) as substrates and has poor activity toward longer peptides. A survey of the literature suggests that the ProAP from A. sobria accepts longer peptides as substrates,¹⁹ but there is otherwise little data available on the substrate specificity of this enzyme. We therefore set out to systematically profile the substrate specificity of A. sobria ProAP by screening a peptide library.

We designed a combinatorial peptide library in the form of PCX¹X²X³X⁴BBRRM-resin (where X¹ X⁴ is any of the 18 canonical amino acids except for cysteine and methionine, aminobutyric acid (Abu) as a cysteine mimetic, or norleucine (Nle) as a methionine mimetic; B=β-alanine), based on the assumption that the enzyme may have sequence selectivity at the P₂′-P₅′ positions (SEQ ID NO: 97). The peptide library was synthesized in the one bead-one compound (OBOC) format²⁰ on the poly(ethyleneglycol-acrylamide) (PEGA) resin (FIG. 6).²¹ The C-terminal methionine permits the peptides to be released from the resin by cyanogen bromide (CNBr) treatment before sequencing analysis by mass spectrometry; substitution of Nle for methionine in the randomized sequence avoids internal peptide cleavage. The arginine residues provide fixed positive charges that increase the sensitivity of detection by mass spectrometry and improve the aqueous solubility of the library peptides. The two β-alanine residues provide a flexible linker, rendering the peptides more accessible to enzymatic action. The library has a theoretical diversity of 160,000 sequences.

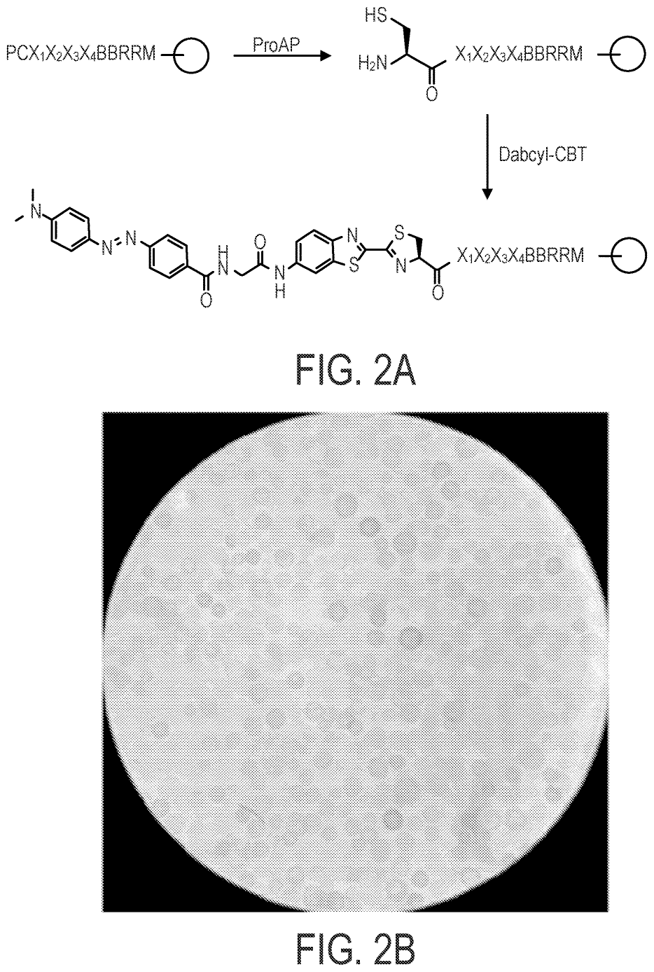

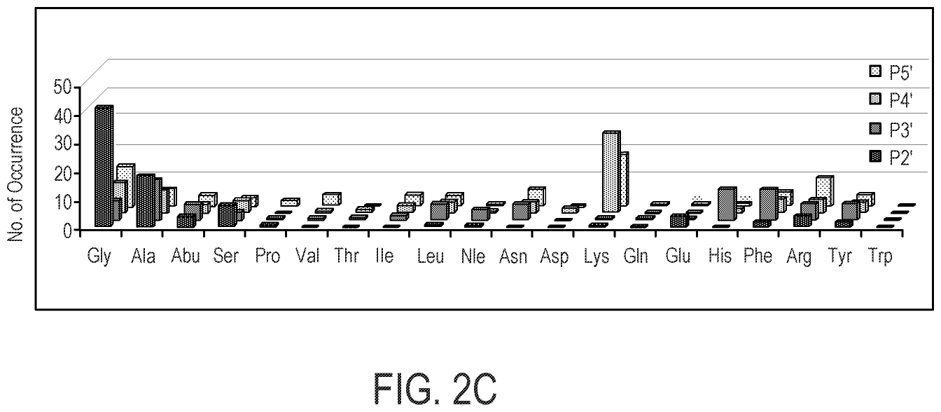

The peptide library was subjected to a limited treatment with A. sobria ProAP (0-1000 nM ProAP for 30 min). Under this competitive condition, only beads displaying the most efficient substrates of A. sobria ProAP underwent partial cleavage of the N-terminal proline, whereas most library beads had little or no reaction. The exposed N-terminal cysteine was next selectively labeled with a Dabcyl-CBT adduct (FIG. 2A). Upon acidification of the reaction solution, “positive” beads that had undergone the most extensive ProAP reaction turned pinkish red while most of the library beads remained colorless. The positive beads were manually isolated from the library using a micropipette with the aid of a dissecting microscope. The peptides were released from the positive beads by cleavage with CNBr and their sequences were determined by MALDI-TOF MS analysis.¹⁵ Under the same screening condition, a control reaction without ProAP resulted in no red-colored bead.

A total of 60 mg of the peptide library (~210,000 beads) was screened against A. sobria ProAP at three different concentrations (at 100, 300, and 1000 nM; 20 mg each). The most intensely colored beads were isolated from the three screening reactions and sequenced to give 28 unambiguous sequences, which represent the most efficient substates of A. sobria ProAP (Table A). An additional 56 sequences were obtained from medium-colored beads (Table B). We also randomly selected 30 colorless beads from the 1000-nM screening experiment and obtained 25 sequences, which represent the poor substrates of A. sobria ProAP (Table C). Similar preferred sequences were obtained from all three screening experiments, demonstrating the reproducibility of the screening method. Inspection of the 84 most efficient sequences revealed that A. sobria ProAP strongly prefers a small residue at the P₂′ position, with Gly being most frequently selected (47% of all selected sequences), followed by Ala (20%) and Ser (9%) (FIG. 2C). At the P₃′ position, the enzyme has some preference for Ala (16%) or aromatic residues such as His (13%) and Phe (12%). At the P₄′ position, Lys (32%) was most frequently selected, followed by Gly (12%) and Ala (9%). The enzyme also shows some preference for positively charged residues at the P₅′ position including Lys (20%) and Arg (12%). On the other hand, most of the poor substrates contained one or more acidic residues (Asp and Glu) while none contained a Gly at the P₂′ position (Table C). Thus, A. sobria ProAP prefers peptides of the consensus Pro-Cys-Gly-(Ala/His)-Lys-Lys.

TABLE A
Peptide Sequences Derived from Intensely Colored
Beads during Library Screening against A. sobria
ProAP (Most Preferred Substrates;
total 28 sequences)^{a} (SEQ ID NOs 98-125
top to bottom, left to right respectively)

| | | | | |
|---|---|---|---|---|
| GNRS | GFKG | AAGQ | ALKH | EFAR |
| GHKF | GGKU | AAAR | ALKN | FGSV |
| GHYA | GFSF | AURU | AAKF | RNYN |
| GGKA^{b} | AALK | ASKA^{b} | SFKG | MIHE |
| GGRA | AHKA^{b} | AFKN | SHKG^{b} | |
| GNFK | ALAR | AGVR | EFTR^{b} | |

^{a}Selected from three screening experiments at 100 nM, 300 nM, and 1.0 μM A. sobria ProAP. U, α-L-aminobutyric acid; M, norleucine,
^{b}Peptides selected for kinetic analysis.

TABLE B
Peptide Sequences Derived from Medium Colored
Beads during Library Screening against A. sobria
ProAP (Preferred Substrates;
total 56 sequences)^{a} (SEQ ID NOS 126-181
top to bottom, left to right respectively)

| | | | | |
|---|---|---|---|---|
| GAGK | GRAU | GFHG | AAGK | PARV |
| GYLS | GHNK | GQUY | UAKS | ERKG |
| GAUR | GYGI | GAFI | UUKN | EIGR |
| GYAF | GNKY | GNKG | UASR | RHTR |
| GHFG | GLKI | GHAG | UMKG | RHFI |
| GFGY | GGRV | GVYG | SLKM | RTNL |
| GMLY | GYUN | GALK | SLYP | KPAK |
| GSSK^{b} | GFIG | AHKK | SUKG | YEML |
| GAIK | GGKF | AHNK | SFNK | YRDU |
| GUGR | GRAL | AUKL | SAKK | FRGK |
| GYSK | GRGA | AMIK | SNKG | LUFN |
| GMGK | | | | |

^{a}Selected from three screening experiments performed at 100, 300, and 1000 nM A. sobria ProAP. U, α-L-aminobutyric acid; M, norleucine.
^{b}Peptide selected for kinetic analysis.

TABLE C
Peptide Sequences Derived from Randomly Selected
Colorless Beads during Library Screening against
A. sobria ProAP (Poor Substrates;
total 25 sequences)^{a} (SEQ ID NOs 182-206
top to bottom, left to right respectively)

| | | | | |
|---|---|---|---|---|
| ADDR | EUVD | HDLA | LQKI | YENI |
| APPE | ELLU | IDIS | VQNI | |
| AMVE | HDET^{b} | IUAU | VNAK | |
| UVPN | HIDS | ITAU | KEUT | |
| DKPK | HAYV | ITNF | QUUI | |
| DFVD | HPQD | LGSP | TUVV | |

^{a}Selected from the screening experiment with 1000 nM A. sobria ProAP. U, α-L-aminobutyric acid; M, norleucine.
^{b}Peptide selected for kinetic analysis.

### Kinetic Properties of Selected ProAP Peptide Substrates

To confirm the screening results, we individually synthesized a panel of peptides and determined their kinetic properties toward A. sobria ProAP (Table 1). Peptides 1-6 are representative (preferred) sequences selected from the library, while peptide 7 is the consensus sequence based on the screening results. Peptide 8 is a variant of peptide 7, containing a proline (instead of a lysine) at the P₅′ position. We anticipated that a proline residue at this position would improve the proteolytic stability of the peptide. Peptide 9 is a variant of peptide 6 (substitution of Gly for Ala at the P₂′ position) and was designed to test the importance of a Gly residue at the P₂′ position. Peptide 10 is also a variant of peptide 6 but contains a Pro at position P₅′ (in place of Lys) to impart proteolytic stability. Peptide 11 is derived from a colorless bead during library screening (Table C) and expected to be a poor substrate of ProAP (“negative” control). All peptides contained a Tyr at the P₆′ position, to facilitate concentration measurement as well as monitoring the enzymatic reactions by UPLC (at 280 nm).

TABLE 1
Kinetic constants of A. sobria ProAP against
selected peptide substratesª (SEQ ID
NOs 207-217 top to bottom respectively)

| Peptide ID | Sequence | k_{cat}/K_{M} (×10⁵ M⁻¹ s⁻¹) |
|---|---|---|
| 1 | PCGGKAY | 7.0 ± 1.2 |
| 2 | PCGSSKY | 5.8 ± 1.0 |
| 3 | PCASKAY | 3.8 ± 0.4 |
| 4 | PCSHKGY | 3.2 ± 0.1 |
| 5 | PCEFTRY | 3.2 ± 0.9 |
| 6 | PCAHKAY | 2,9 ± 0.4 |
| 7 | PCGHKKY | 8.2 ± 0.7 |
| 8 | PCGHKPY | 8.3 ± 3.2 |
| 9 | PCGHKAY | 7.2 ± 2.6 |
| 10 | PCAHKPY | 2.8 ± 0.8 |
| 11 | PCHDETY | 0.038 ± 0.010 |

ªAll peptides contained a free N-terminus and a C-terminal amide. Values reported represent the mean ± SD of three independent sets of experiments.

Peptides 1-10 are highly efficient substrates of A. sobria ProAP, having k_{cat}/K_{M} values of 2.8-8.3×10⁵ M⁻¹s⁻¹ (Table 1). However, most of the substrates did not saturate the enzyme even at the highest substrate concentration tested (500 μM), suggesting that A. sobria ProAP has high K_{M} values toward peptidyl substrates. As such, we were only able to determine the k_{cat}/K_{M} values of these substrates, but not their k_{cat} or K_{M} values. The kinetic data confirmed that the P₂′ position is a critical specificity determinant for A. sobria ProAP, with Gly being the most preferred amino acid. Even substitution of Ala, which is the second most preferred residue at this position, decreased the catalytic activity by 2- to 3-fold (compare peptides 6 and 9 or peptides 8 and 10). In contrast, the P₃′ to P₅′ positions play a more minor role in substrate recognition and tolerate a variety of amino acids. Replacement of a His at the P₃′ position with Gly or Ser altered the catalytic activity by only ≤1.3-fold (compare peptides 1 and 9 or peptides 3 and 6). A positively charged residue (Lys or Arg) at the P₄′ and/or P₅′ position appears to enhance the A. sobria ProAP activity, but the effect is also small (e.g., compare peptides 7-9). Importantly, peptide 11 (the “negative” control) is ~2 orders of magnitude less active than peptides 1-10, highlighting the importance of a proper peptide sequence for optimal A. sobria ProAP activity.

### N-Terminal Conjugation of Peptides

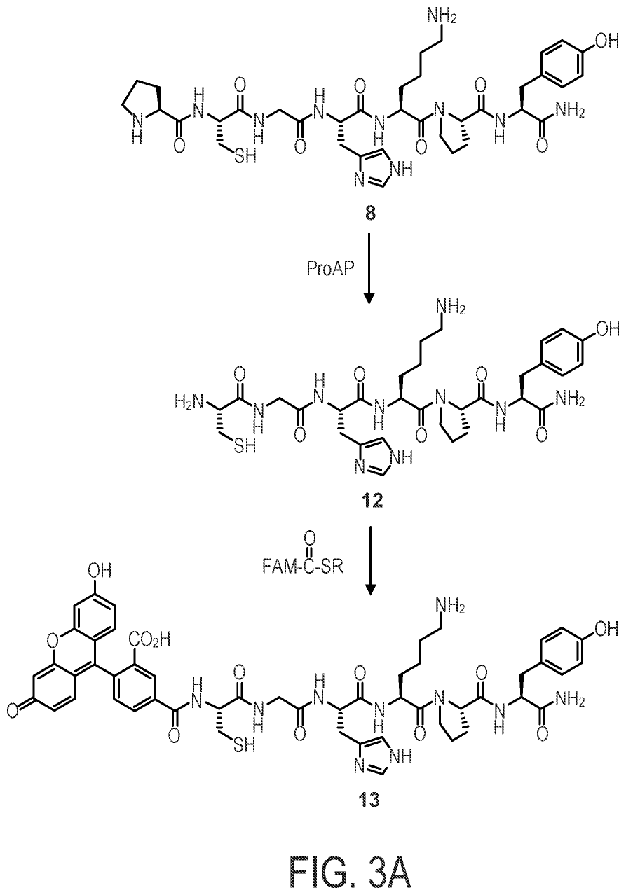

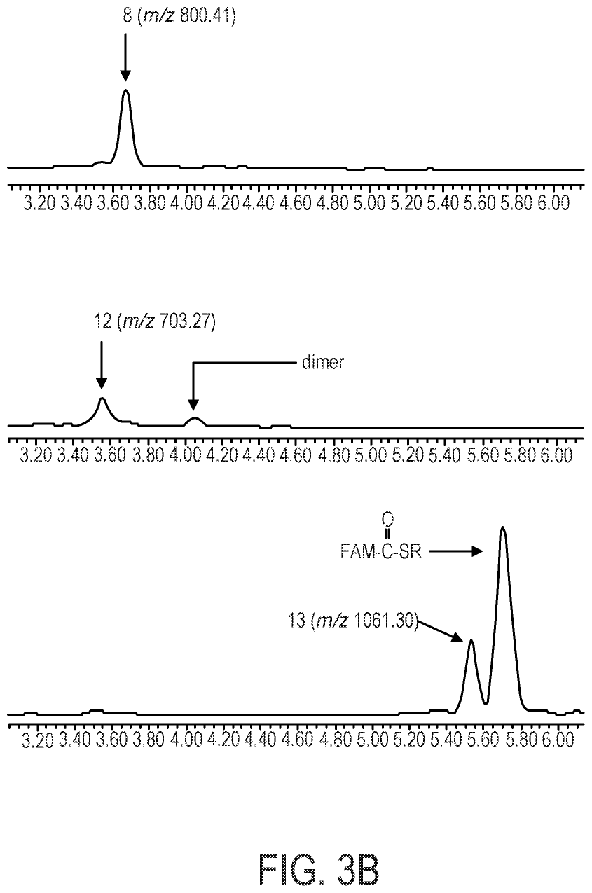

We first tested the feasibility of generating an N-terminal cysteine by ProAP and specific N-terminal conjugation with a peptide as substrate. Treatment of peptide 8 (100 μM) with A. sobria ProAP (4 μM) for 60 min resulted in the complete disappearance of peptide 8 (retention time=3.66 min on UPLC) and the formation of a new peak at 3.56 min (peptide 12) (FIGS. 3A-3B). A small peak with a retention time of 4.05 min was also formed; this peak is due to the formation of a small amount of disulfide-bonded peptide dimer, as no reducing agent was included in the ProAP reaction [tris(carboxyethyl)phosphine (TCEP) strongly inhibits the A. sobria ProAP activity]. The reaction mixture was next treated with an excess of fluorescein 2-mercaptoethanesulfonate thioester (FAM-CO-SR; 4 equivalents) in the presence of 2-mercaptoethanesulfonate (2-MES) and TCEP in a phosphate buffer (pH 7.5). This procedure resulted in the total loss of both monomeric and dimeric peaks of peptide 12 and the formation of a new peak at 5.53 min, which has a m/z value of 1061.3, consistent with the addition of a fluorescein to the N-terminus of peptide 12.

### N-Terminal Conjugation of Proteins

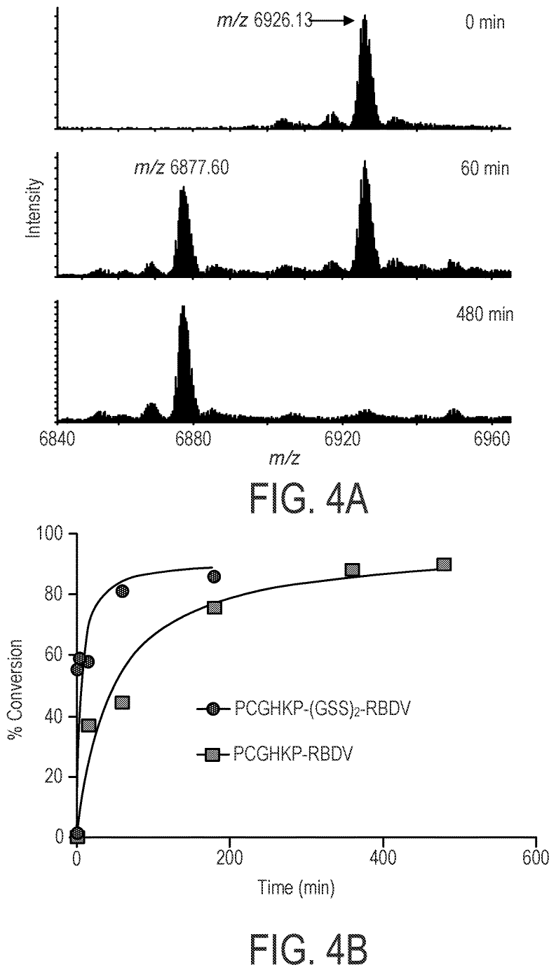

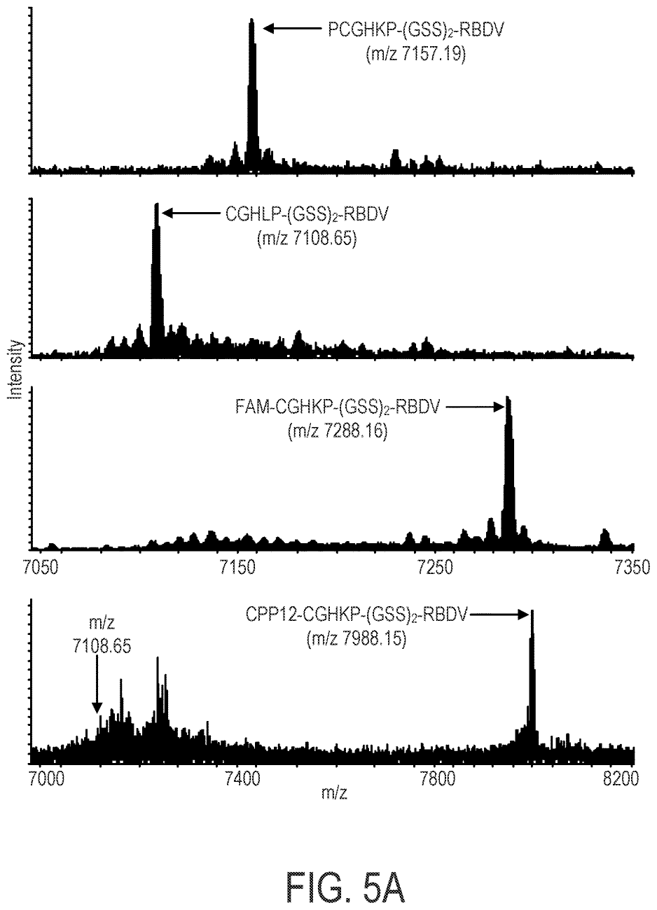

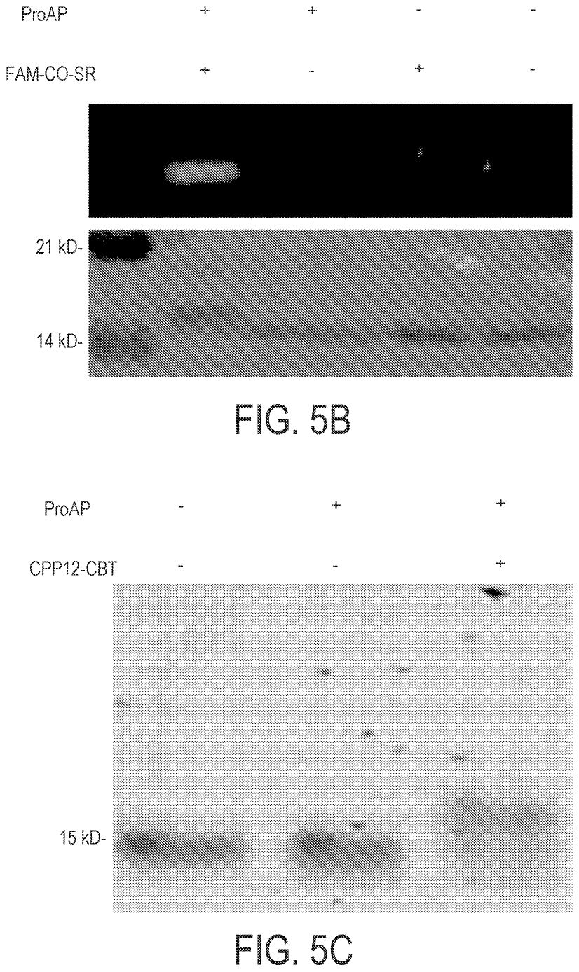

We chose an engineered variant of the Ras-binding domain (RBD) of c-Raf, RBDV22, for N-terminal conjugation. RBDV (SEQ ID NO: 87) binds selectively to the GTP-bound (activated) form of Ras with high affinity (K_{D}=3 nM for K-Ras). Furthermore, intracellular delivery of RBDV (SEQ ID NO: 87) may potentially provide a novel treatment for Ras mutant cancers. We genetically fused the optimal substrate sequence of A. sobria ProAP, PCGHKP (SEQ ID NO. 218), to the N-terminus of RBDV. Treatment of PCGHKP-RBDV (25 μM) (SEQ ID NO: 95) with ProAP (1 μM) resulted in the removal of the N-terminal proline, as indicated by the conversion of the m/z 6926.13 species ([M+2H]²⁺) into a new species at m/z 6877.60 in high-resolution mass spectrometry (FIG. 4A). However, the enzymatic reaction was sluggish and required 6 h to complete (t_{1/2}~1 h) (FIG. 4B). Given the robust activity of A. sobria ProAP against peptidyl substrates (Table 1), we reasoned that the N-terminally fused ProAP recognition motif may be sterically hindered by the RBDV structure (SEQ ID NO: 87). We therefore generated a second construct in which a flexible, hydrophilic, and proteolytically stable linker, (GSS)₂, was inserted between the PCGHKP sequence (SEQ ID NO. 218) and the RBDV structure (SEQ ID NO: 87). The presence of a flexible linker should also minimize the formation of secondary structures by the PCGHKP motif (SEQ ID NO. 218) and any potential interference with the folding and/or function of the protein of interest.²³ Gratifyingly, PCGHKP-(GSS)₂-RBDV (SEQ ID NO: 96) is a greatly improved substrate of A. sobria ProAP, undergoing complete proline removal within 60 min (t_{1/2}~1 min) (FIGS. 4B and 5A).

The ProAP reaction product, CGHKP-(GSS)₂-RBDV (m/z 7108.65) (SEQ ID NO. 219), was next treated with an excess (8 equivalents) of thioester FAM-CO-SR in a phosphate buffer (pH 7.5). The m/z 7108.65 peak was slowly converted into a new peak at m/z 7288.16, which is consistent with the addition of a fluorescein to the N-terminus of RBDV (FIG. 5A) (SEQ ID NO: 87). Based on the ratio of peak intensities, it was estimated that 93% (+1%) of CGHKP-(GSS)₂-RBDV was converted into FAM-CGHKP-(GSS)₂-RBDV after 5 h. SDS-PAGE analysis of the reaction products showed a single fluorescent band of ~15 kDa and successful protein labeling required the presence of both ProAP and FAM-CO-SR (FIG. 5B).

Finally, we conjugated a highly efficient cyclic cell-penetrating peptide, cyclo(Phe-phe-Nal-Arg-arg-Arg-arg-Gln) (CPP12, where arg is D-arginine, phe is D-phenylalanine, and Nal is L-naphthylalanine), to the N-terminus of RBDV (SEQ ID NO: 87) through a CBT moiety. Treatment of CGHKP-(GSS)₂-RBDV (SEQ ID NO. 219) with 5 equivalents of CPP12-CBT in phosphate buffered saline (pH 7.4) for 2 h resulted in the formation of a new species of higher molecular weight (~16 kDa) (FIG. 5C). MALDI-FT-ICR MS analysis of the reaction mixture showed nearly complete loss of the m/z 7108.65 peak and the concomitant formation of a new peak at m/z 7988.15 (FIG. 5A). The latter is consistent with the [M+2H]²⁺ ion of a CPP12-CGHKP-(GSS)₂-RBDV (SEQ ID NO. 220) adduct. Intracellular delivery of the CPP12-RBDV (SEQ ID NO. 221) conjugate will be the subject of future studies.

### CONCLUSION

This example provides a method to produce recombinant proteins containing an N-terminal cysteine, which can be site-specifically modified to install various functional entities by reacting with thioesters to form amides (native chemical ligation), CBT to form 2-thiazolines, or aldehydes to form thiazolidines. A key advantage is that ProAP only removes a proline from the N-terminus of a protein without causing nonspecific cleavage anywhere else in the protein or further N-terminal cleavage of the reaction product. This permits the use of excess ProAP activity and/or extended reaction time (if necessary) to drive the intended reaction (i.e., the removal of N-terminal proline) to completion. In contrast, endopeptidases (e.g., TEV, thrombin, and factor Xa) are not completely sequence specific and often cause cleavage at unintended sites in proteins, especially under forcing conditions or when the intended cleavage site is hindered by the formation of secondary structures. Although this example only tested our method on bacterially expressed proteins, it should also be applicable to proteins produced in eukaryotic cells, e.g., the generation of antibody-drug conjugates. On the other hand, previous methods that utilize the endogenous N-terminal processing enzymes (e.g., MetAP and leader peptidase) are only effective for proteins produced in bacteria, as proteins produced in eukaryotes are usually N-terminally acetylated. A minor drawback is that a short ProAP recognition motif (CGHKP (SEQ ID NO: 223) or CGHKPGSSGSS (SEQ ID NO: 224)) is retained in the final protein product, which may not be compatible with all applications.

### REFERENCES

(1) Lieser, R. M.; Yur, D.; Sullivan, M. O.; Chen, W. Site-specific bioconjugation approaches for enhanced delivery of protein therapeutics and protein drug carriers. Bioconjug. Chem. 2020, 3/, 2272-2282.
(2) Somnay, C.; Vaur, V.; Wagner, A.; Chaubet, G. An overview of chemo- and site-selectivity aspects in the chemical conjugation of proteins. Royal Society Open Science 2022, 9, 211563.
(3) Rosen, C. B.; Francis, M. B. Targeting the N terminus for site-selective protein modification. Nat. Chem. Biol. 2017, 13, 697-705.
(4) Zhang, L.; Tam, J. P. Thiazolidine formation as a general and site-specific conjugation method for synthetic peptides and proteins. Anal. Biochem. 1996, 233, 87-93.
(5) Dawson, P. B.; Muir, T. W.; Clark-Lewis, I., Kent, S. B. H. Synthesis of proteins by native chemical ligation. Science 1994, 266, 776-779.
(6) Ren, H.; Xiao, F.; Zhan, K.; Kim, Y. P.; Xie, H.; Xia, Z., Rao, J. A biocompatible condensation reaction for the labeling of terminal cysteine residues on proteins. Angew. Chem. Int. Ed. Engl. 2009, 48, 9658-9662.
(7) Gentle, I. E.; De Souza, D. P.; Baca, M. Direct production of proteins with N-terminal cysteine for site-specific conjugation. Bioconjug. Chem. 2004, 15, 658-663.
(8) Giglione, C.; Boularot, A.; Meinnel, T. Protein N-terminal methionine excision. Cell. Mol. Life S. 2004, 61, 1455-1474.
(9) Ree, R., Varland, S.; Arnesen, T. Spotlight on protein N-terminal acetylation. Exp. Mol. Med. 2018, 50, 1-13.
(10) Hauser, P. S.; Ryan, R. O. Expressed protein ligation using an N-terminal cysteine containing fragment generated in vivo from a pelB fusion protein. Protein Expression and Purification 2007, 54, 227-233.
(11) Freudl, R. Signal peptides for recombinant protein secretion in bacterial expression systems. Microb. Cell Factories 2018, 17, 52.
(12) Low, K. O.; Muhammad Mahadi, N.; Illias, R. Optimisation of signal peptide for recombinant protein secretion in bacterial hosts. Appl. Microbiol. Biotechnol. 2013, 97, 3811-3826.
(13) Tolbert, T. J.; Wong, C. H. New methods for proteomic research: preparation of proteins with N-terminal cysteines for labeling and conjugation. Angew. Chem., Int. Ed. 2002, 41, 2171-2174.
(14) Jenny, R. J.; Mann, K. G.; Lundblad, R. L. A critical review of the methods for cleavage of fusion proteins with thrombin and factor Xa. Protein Expression and Purification 2003, 37, 1-11.
(15) Hu, Y. J.; Wei, Y.; Zhou, Y.; Rajagopalan, P. T.; Pei, D. Determination of substrate specificity for peptide deformylase through the screening of a combinatorial peptide library. Biochemistry 1999, 38, 643-650.
(16) Hirel, P. H.; Schmitter, M. J.; Dessen, P.; Fayat, G.; Blanquet, S. Extent of N-terminal methionine excision from Escherichia coli proteins is governed by the side-chain length of the penultimate amino acid. Proc. Natl. Acad. Sci. U.S.A. 1989, 86, 8247-8251.
(17) Xiao, Q.; Zhang, F.; Nacev, B. A.; Liu, J. O., Pei, D. Protein N-terminal processing: substrate specificity of Escherichia coli and human methionine aminopeptidases. Biochemistry 2010, 49, 5588-5599.
(18) Yoshimoto, T.; Tsuru, D. Proline iminopeptidase from Bacillus coagulans: purification and enzymatic properties. J. Biochem. 1985, 97, 1477-1485.
(19) Kitazono, A.; Kitano, A.; Tsuru, D.; Yoshimoto, T. Isolation and characterization of the prolyl aminopeptidase gene (pap) from Aeromonas sobria: comparison with the Bacillus coagulans enzyme. J. Biochem. 1994, 116, 818-825.
(20) Lam, K. S.; Salmon, S. E.; Hersh, E. M.; Hruby, V. J.; Kazmierski, W. M.; Knapp, R. J. A new type of synthetic peptide library for identifying ligand-binding activity. Nature 1991, 354, 82-84.
(21) Auzanneau, F.-L; Meldal, M., Bock, K. Synthesis, characterization and biocompatibility of PEGA resins. J. Pept. Sci. 1995, 1, 31-44.
(22) Wiechmann, S.; Maisonneuve, P.; Grebbin, B. M.; Hoffmeister, M., Kaulich, M.; Clevers, H.; Rajalingam, K.; Kurinov, I.; Farin, H. F.; Sicheri, F.; Ernst, A. Conformation-specific inhibitors of activated Ras GTPases reveal limited Ras dependency of patient-derived cancer organoids. J. Biol. Chem. 2020, 295, 4526-4540.
(23) Van Rosmalen, M.; Krom, M.; Merkx, M. Tuning the flexibility of glycine-serine linkers to allow rational design of multidomain proteins. Biochemistry 2017, 56, 6565-6574,
(24) Youngquist, R. S.; Fuentes, G. R.; Lacey, M. P.; Keough, T. Generation and screening of combinatorial peptide libraries designed for rapid sequencing by mass spectrometry. J. Am. Chem. Soc. 1995, 117, 3900-3906.
(25) Qian, Z.; Martyna, A., Hard, R. L.; Wang, J.; Appiah-Kubi, G.; Coss, C.; Phelps, M. A.; Rossman, J. S.; Pei, D. Discovery and mechanism of highly efficient cyclic cell-penetrating peptides. Biochemistry 2016, 55, 2601-2612.
(26) Qi, D.; Scholthof, K. B. G. A one-step PCR-based method for rapid and efficient site-directed fragment deletion, insertion, and substitution mutagenesis. J. Virol. Methods 2008, 149, 85-90

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A method of preparing a peptide having an N-terminal cysteine residue of Formula I:
Cys-[peptide] (I)
wherein Cys is the N-terminal cysteine residue and [peptide] comprises an amino acid sequence having at least five residues;
the method comprising:
(i) expressing within a prokaryotic cell a first peptide of Formula II-a:
fMet-Pro-Cys-[peptide] (II-a)
wherein fMet is an N-formylmethionine residue and Pro is a proline residue, and
wherein the prokaryotic cell further expresses a peptide deformylase (PDF) and a methionine amino peptidase (MetAP) such that the fMet residue is cleaved in situ to provide a second peptide of Formula III:
Pro-Cys-[peptide] (III);
(ii) isolating the second peptide from the prokaryotic cell;
(iii) contacting the second peptide of Formula III with a prolyl aminopeptidase (ProAP) to provide the peptide of Formula I.

2. (canceled)

3. The method of claim 1, wherein the prokaryotic cell comprises an Escherichia coli cell or a Bacillus subtilis cell.

4. The method of claim 1, wherein [peptide] further comprises a recognition sequence for the ProAP.

5. The method of claim 4, wherein the recognition sequence comprises a sequence selected from SEQ ID NO. 1 to SEQ ID NO. 84.

6. The method of claim 1, wherein the ProAP comprises a ProAP isolated from Aeromonas sobria, Aspergillus oryzae, Aspergillus niger, or Debaryomyces hansenii.

7. The method of claim 1, wherein the first peptide is expressed from at least one non-naturally occurring gene within the cell.

8. The method of claim 7, wherein the at least one non-naturally occurring gene is integrated into the genome of the cell or into an expression vector.

9-10. (canceled)

11. A method of preparing a peptide of Formula I:
Cys-[peptide] (I)
wherein Cys comprises a cysteine residue at the N-terminus and [peptide] comprises a peptide sequence having at least five amino acid residues;
the method comprising:
(i) expressing within a eukaryotic cell a first peptide of Formula II-b:
Met-Pro-Cys-[peptide] (II-b)
wherein Met comprises a methionine residue and Pro comprises a proline residue,
wherein the cell further expresses a methionine amino peptidase (MetAP) such that the Met residue is cleaved in situ to provide a second peptide of Formula III:
Pro-Cys-[peptide] (III);
(ii) isolating the second peptide from the eukaryotic cell;
(iii) contacting the second peptide of Formula III with a prolyl aminopeptidase to provide the peptide of Formula I.

12. (canceled)

13. The method of claim 11, wherein [peptide] further comprises a recognition sequence for the ProAP.

14. The method of claim 13, wherein the recognition sequence comprises a sequence selected from SEQ ID NO. 1 to SEQ ID NO. 84.

15. The method of claim 11, wherein the wherein the ProAP comprises a ProAP isolated from Aeromonas sobria, Aspergillus oryzae, Aspergillus niger, or Debaryomyces hansenii.

16. The method of claim 11, wherein the first peptide is expressed from at least one non-naturally occurring gene within the cell.

17. The method of claim 16, wherein the at least one non-naturally occurring gene is integrated into the genome of the cell or into an expression vector.

18-19. (canceled)

20. A peptide prepared according to the method of claim 1.

21. A method for preparing a chemically-modified peptide, the method comprising:
(i) preparing a peptide having an N-terminal cysteine residue according to the method of claim 1; and
(ii) conjugating the peptide with a ligand, wherein the ligand comprises a moiety capable of reacting with the N-terminal cysteine residue.

22-23. (canceled)

24. A chemically-modified peptide prepared according to the method of claim 21.

25. A method for determining the specificity of a prolyl aminopeptidase (ProAP) comprising:
(i) providing a library of peptides, wherein each peptide has an N-terminal sequence comprising Pro-Cys, wherein Pro is an N-terminal proline and Cys is cysteine;
(ii) contacting the library of peptides with the prolyl aminopeptidase such that Pro is cleaved in a portion of the library of peptides to provide a cleaved portion;
(iii) contacting the library of peptides with a label having a moiety capable of reacting with an N-terminal cysteine residue such that the cleaved portion is labeled to provide a labeled portion;
(iv) isolating the labeled portion; and
(v) sequencing the labeled portion.

26-29. (canceled)

30. A peptide prepared according to the method of claim 11.

31. A method for preparing a chemically-modified peptide, the method comprising:
(i) preparing a peptide having an N-terminal cysteine residue according to the method of claim 11; and
(ii) conjugating the peptide with a ligand, wherein the ligand comprises a moiety capable of reacting with the N-terminal cysteine residue.

32. A chemically-modified peptide prepared according to the method of claim 31.
